SEQUENCE LISTING (11 sequences)

SEQ ID NO: 1 - protein, 504 aa, Homo sapiens
MSPSPTALFCLGLCLGRVPAQSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTGTSVTPSRLPTEPPSSVAEFSEATAELTVSFTNKVFTTETSRSITTSPKESDSPAGPARQYYTKGNGGRESKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 318 aa, Artificial Sequence
  Signal peptide / Extracellular Domain / Transmembrane and cytoplasmic domains Cynomolgus monkey GPVI
MSPSPTTLFCLGLCLGHVPAQRGPLPKPSLQALPSSLVPLEKPVTLRCQGPPGVDLYRLEKLSSSRYQDQAVLFIPAMKRHLAGRYRCSYQNGSLWSPPSDQLELVATGVFAKPSLSAQPGPAVSSGGDVTLQCQTRYGFDQFALYKEGDPAPYKNPERWYRASFPIITVTAAHSGTYRCYSFSSGDPYLWSAPSDPLELMVTEFSEATTELTVSLTNKVFTTETSRSITASPKEPGSPAGPARQYYTKGNLVRICLGAVILILLAGFLAEDWHSRRKRLRHRVRAVQRPLPPLPPTRKSHGDQDGGRPDVHSRGLCS

SEQ ID NO: 3 - protein, 313 aa, Mus musculus
MSPASPTFFCIGLCVLQVIQTQSGPLPKPSLQAQPSSLVPLGQSVILRCQGPPDVDLYRLEKLKPEKYEDQDFLFIPTMERSNAGRYRCSYQNGSHWSLPSDQLELIATGVYAKPSLSAHPSSAVPQGRDVTLKCQSPYSFDEFVLYKEGDTGSYKRPEKWYRANFPIITVTAAHSGTYRCYSFSSSSPYLWSAPSDPLVLVVTGLSATPSQVPTEESFPVTESSRRPSILPTNKISTTEKPMNITASPEGLSPPFGFAHQHYAKGNLVRICLGATIIIILLGLLAEDWHSRKKCLQHRMRALQRPLPPLPLA

SEQ ID NO: 4 - protein, 249 aa, Artificial Sequence
  extracellular domain of human GPVI-1A
QSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTGTSVTPSRLPTEPPSSVAEFSEATAELTVSFTNKVFTTETSRSITTSPKESDSPAGPARQYYTKGN

SEQ ID NO: 5 - protein, 249 aa, Artificial Sequence
  extracellular domain of human GPVI-1B
QSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTGTSVTPSRLPTEPPSPVAEFSEATAELTVSFTNEVFTTETSRSITASPKESDSPAGPARQYYTKGN

SEQ ID NO: 6 - protein, 231 aa, Artificial Sequence
  extracellular domain of human GPVI-2A
QSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTEFSEATAELTVSFTNKVFTTETSRSITTSPKESDSPAGPARQYYTKGN

SEQ ID NO: 7 - protein, 229 aa, Artificial Sequence
  extracellular domain of cynomolgus monkey GPVI
QRGPLPKPSLQALPSSLVPLEKPVTLRCQGPPGVDLYRLEKLSSSRYQDQAVLFIPAMKRHLAGRYRCSYQNGSLWSPPSDQLELVATGVFAKPSLSAQPGPAVSSGGDVTLQCQTRYGFDQFALYKEGDPAPYKNPERWYRASFPIITVTAAHSGTYRCYSFSSGDPYLWSAPSDPLELMVTEFSEATTELTVSLTNKVFTTETSRSITASPKEPGSPAGPARQYYTK

SEQ ID NO: 8 - protein, 246 aa, Artificial Sequence
  extracellular domain of mouse GPVI
QSGPLPKPSLQAQPSSLVPLGQSVILRCQGPPDVDLYRLEKLKPEKYEDQDFLFIPTMERSNAGRYRCSYQNGSHWSLPSDQLELIATGVYAKPSLSAHPSSAVPQGRDVTLKCQSPYSFDEFVLYKEGDTGSYKRPEKWYRANFPIITVTAAHSGTYRCYSFSSSSPYLWSAPSDPLVLVVTGLSATPSQVPTEESFPVTESSRRPSILPTNKISTTEKPMNITASPEGLSPPFGFAHQHYAKGN

SEQ ID NO: 9 - protein, 248 aa, Artificial Sequence
  extracellular domain of rat GPVI
QHGPLPKPSLQAQPSSLVPLGHPVTLRCLGPSDADLYRLEKVKPGKLIFIDQDFLFIPIMEINNAGRYRCSYQNESHWSLPSDQLELIATGVYSKPSLSAHPSSAIPPGRDVTLKCQSQYSFDEFVLYKEGDTRPYKRPEKWYRANFPVITVTAAHSGTYRCYSFSSSSPYLWSAPSDPLVVVVTGPSATPSQVPTEVPSPMTEASRRPSMLLTNKISTTEKPMNITVSPEGPSPPFGFAHQHYAKGN

SEQ ID NO: 10 - protein, 228 aa, Homo sapiens
DIKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 339 aa, Homo sapiens
MSPSPTALFCLGLCLGRVPAQSGPLPKPSLQALPSSLVPLEKPVTLRCQGPPGVDLYRLEKLSSSRYQDQAVLFIPAMKRSLAGRYRCSYQNGSLWSLPSDQLELVATGVFAKPSLSAQPGPAVSSGGDVTLQCQTRYGFDQFALYKEGDPAPYKNPERWYRASFPIITVTAAHSGTYRCYSFSSRDPYLWSAPSDPLELVVTGTSVTPSRLPTEPPSSVAEFSEATAELTVSFTNKVFTTETSRSITTSPKESDSPAGPARQYYTKGNLVRICLGAVILIILAGFLAEDWHSRRKRLRHRGRAVQRPLPPLPPLPLTRKSNGGQDGGRQDVHSRGLCS

(19) United States
(12) Patent Application Publication
(10) Pub. No.: US 2025/0018010 A1
(43) Pub. Date: Jan. 16, 2025

(54) PHARMACEUTICAL COMPOSITION

(71) Applicant: advanceCOR GmbH, Planegg (DE)

(72) Inventors: Götz Münch, Munich (DE); Kristin Adler, Munich (DE); Meinrad Gawaz, Tübingen (DE)

(73) Assignee: advanceCOR GmbH, Planegg (DE)

(21) Appl. No.: 18/701,328

(22) PCT Filed: Jun. 10, 2022
(86) PCT No.: PCT/EP2022/065901

§ 371 Date: Apr. 15, 2024

(30) Foreign Application Priority Data
Jun. 18, 2021 (EP) 21 180 388.7

(51) Int. Cl. A61K 38/17; A61K 45/06; A61K 47/18; A61K 47/26; A61P 7/02

(52) U.S. Cl. CPC A61K 38/1774; A61K 45/06; A61K 47/183; A61K 47/26; A61P 7/02

(57) ABSTRACT

A pharmaceutical composition for parenteral application. comprising (a) more than 2.4 mg/ml of a collagen-binding dimeric fusion protein comprising an extracellular domain of glycoprotein VI, the fusion protein having an amino acid sequence of SEQ ID NO: 1, or an amino acid sequence that is at least 99% homologous to the amino acid sequence of SEQ ID NO: 1; and (b) a pharmaceutically acceptable buffer, the buffer comprising (b1) a buffering component in combination with a ph adjusting agent, which is 10 mM His/HCl, and (b2) 4 percent by weight of mannitol and (b3) 2.5 percent by weight of saccharose as stabilizing agents, and which has a pH of 7

### FIELD OF THE INVENTION

The present invention relates to a pharmaceutical composition for parenteral application. Specifically, said pharmaceutical composition comprises a collagen-binding dimeric fusion protein comprising an extracellular domain of glycoprotein VI and a specific buffer. The present invention also relates to a lyophilized composition adapted to provide a pharmaceutical composition according to the present invention. The pharmaceutical composition for parenteral application according to the present invention may be prepared with an increased concentration of the pharmaceutically active ingredient while having improved storage stability, which avoids administering large volumes of the pharmaceutical composition to be administered and cooling of the pharmaceutical composition to low temperatures during storage.

Moreover, the present invention relates to a pharmaceutical composition containing a collagen-binding dimeric fusion protein comprising an extracellular domain of glycoprotein VI for use in the prevention or treatment of bleeding complications in a patient specifically selected based on a biomarker. According to the present invention, a diagnostic method using an antibody may be used to select patients in need of a reduction of the collagen-induced platelet stress by administering a collagen-binding dimeric fusion protein. The present invention also relates to a kit-of-parts comprising a pharmaceutical composition of the present invention and a diagnostic antibody as well as a diagnostic method for selecting a patient benefitting from administration of a dimeric fusion protein comprising an extracellular domain of glycoprotein VI for preventing bleeding complications.

### BACKGROUND

Gador, Eva “Strategies to improve the biological performance of protein therapeutics”, Dissertation zur Erlangung des naturwissenschaftlichen Doktorgrades der Justus Maximillian Universität Würzburg (2017) pages 13 to 199, XP055869441 discloses Revacept® as a solution of 40 mg PR-15 per vial for intravenous application. PR-15 is a dimeric fusion protein composed of the extracellular domain of human glycoprotein VI (GP VI, a platelet specific adhesion receptor), a short hinge region and the Fc region of human immunoglobulin G1 (IgG1) (FIG. 17B). Two monomers of the fusion protein are covalently linked via intermolecular disulfide bonds in the hinge region. PR-15 is a lesion specific platelet adhesion inhibitor which prevents arterial thrombosis with the same efficacy as known antiplatelet agents but without bleeding risk. PR-15 consists of 483 naturally occurring amino acids (FIG. 17A). The signal sequence (amino acids 1-20) is cleaved off. Amino acids from 21 to 269 are involved in binding to the GP VI ligand collagen. The polypeptide sequence of the Fc part of the fusion protein covers the residues 275 to 503.

Platelet activation is of fundamental importance in the development of arterial thrombosis and cardiovascular disorders. Patients suffering from such disorders are commonly treated with antiplatelet drugs which interfere with thrombus formation through targeting late events in this process. A serious side effect of these drugs is prolonged bleeding which limits their use.

Glycoprotein VI (GPVI) is a major collagen receptor expressed exclusively on platelets and megakaryocytes. Binding of GPVI to collagen (which is one of the most important thrombogenic components of the subendothelial matrix (Lockyer S. et al, Thromb Res. 2006; 1 18(3):371-80)) induces receptor clustering and subsequent platelet activation. As such, GPVI is of central importance in early events of platelet activation, and therefore a major target for the interference with this mechanism (Nieswandt B and Watson SP, Blood. 2003 Jul. 15; 102(2):449-61). The antiplatelet and antithrombotic effects of GPVI have been described in in vitro and in vivo studies, using platelets from mice and human. For instance, GPVI deficient platelets do not respond to collagen. Moreover, mice deficient for GPVI revealed an effective inhibition of arterial thrombus formation at the damaged vessel wall without increasing the susceptibility to spontaneous bleeding. All these data indicate that GPVI is an effective and safe target for the treatment of thrombotic and vascular disorders in human.

GPVI-mediated and collagen-bound von Willebrand Factor (vWF)-dependent platelet adhesion and activation play important roles in human plaque-triggered thrombus formation and subsequent development of cardiovascular syndromes such as stroke. GPVI expression is specifically observed in platelets and megakaryocytes.

The interaction of GPVI with collagen can be inhibited competitively by a dimeric GPVI-Fc fusion protein (Revacept®) or by antibodies which have been developed to block GPVI. Dimeric soluble glycoprotein VI (GPVI-Fc) inhibits platelet-induced thrombus formation at sites of vascular injury. Administration of GPVI-Fc improves myocardial ischemia and cerebral infarction without affecting bleeding time and inhibits progression of atherosclerosis. GPVI-Fc also inhibits collagen-induced aggregation in humans in a phase I study. GPVI-Fc acts locally at the site of plaque rupture and is most effective under high shear flow. Dimeric GPVI-Fc (Revacept®) binds to GPVI binding sites on plaque collagen without increasing bleeding in clinical studies.

Despite the potent inhibition activity against platelet aggregation, the dimeric GPVI-Fc fusion protein (Revacept®) has a limited storage stability and must be provided in highly diluted dispersion. For example, the fusion protein must be kept at low concentration to avoid aggregation. Consequently, the large volume of the solvent makes the formulation difficult to use. In one aspect, a liquid formulation containing a typical dosage of fusion protein requires a large volume of diluent. The resulting formulation cannot be filled into a single injector commonly used. Furthermore, a large volume of solvent in a frozen liquid formulation also requires a long time for thawing or warming. Furthermore, the formulation of said fusion protein requires a low temperature for long term storage. A typical storage temperature is −80° C., which limits the distribution of the fusion protein due to the burdened requirement for storage and transportation. Moreover, the low storage temperature further prolongs the time required to thaw and reconstitute the fusion protein for parenteral application.

Moreover, bleeding complications are the single event with the most serious impact on the further clinical outcome of patients undergoing cardiac or other vascular interventions (Ndrepepa et al, J Am Coll Cardiol 2008; 51:690-7). A personalized drug therapy for antiplatelet and anticoagulant drugs would help to reduce bleeding complications. Therefore, many efforts have been made to have reliable predictions for the further outcome of these vulnerable patients. The most widely prognostic test for bleeding is the clinical risk stratification with different risk factors. However, today a confusing multitude of different stratification schemes are used suggesting a failure to sufficiently predict bleedings by now. Additional functional tests such as platelet functions tests have been additionally used to determine either the risk of future ischemic events due to variability of platelet inhibition or the future risk of bleeding. Most of these tests are difficult to handle and highly dependent on blood sampling and preparation. Additionally, only fresh whole blood with no possibility for storage or longer transportation is required for reproducible results. However, technical improvements as the multiplate system using whole blood allowed for predictive results for ischemic complications by identifying low-responders to platelet inhibition therapy. The multiplate aggregation was able to predict the occurrence of 30 day stent thrombosis with 70% sensitivity and 84% specificity in patients with insufficient reaction to clopidogrel. Very robust inhibition of platelet aggregation (lowest quintile of platelet aggregation), was unable to predict the future bleedings in these patients. Nevertheless, more recently cut-off values for high and low platelet reactivity were defined for an optimal corridor of platelet aggregation in a consensus paper on platelet aggregation tests (Tantry US et al, J Am Coll Cardiol 2013; 62: 2261-73.). The authors however stated that a future trial is mandatory to validate the therapeutic window.

A robust hands-on assay is desirable to determine the future risk of patients depending on the degree of platelet activation is, therefore, desired, which allows to avoid either the over-treatment with increased bleeding complications or under-treatment with overt ischemic complications such as stent thrombosis.

Platelet activation by collagen as a component of the atherosclerotic plaque via the main collagen receptor Glycoprotein VI (GPVI) on the surface of platelets is considered the crucial mechanism for arterial thrombosis leading to myocardial infarction or stroke. During this activation process a part of the GPVI is cleaved from the surface of the platelet and released into the blood by a metalloproteinase dependent process. The amount of this soluble form of GPVI (sGPVI) can be determined in the patients' blood. The hypothesis is that number and amount of cleaved sGPVI correlates with the degree of platelet activation. However, the previous studies on sGPVI for the prediction of ischemic or bleeding events have been inconclusive. More recently, cleavage of the GPVI receptor on platelets and the consecutive increase of sGPVI in the blood has been associated with reduced platelet function and increased bleeding especially in trauma patients (Vulliamy et al, Blood advances 2020; 4 (12): 2623-2630).

### SUMMARY OF THE INVENTION

There is thus a need for a pharmaceutical composition comprising a collagen-binding fusion protein which is suitable for parenteral application and improved in terms of storage stability, efficacy and safety profiles in human.

Accordingly, the present invention provides novel pharmaceutical composition which is superior to formulations known from the prior art. In general, the present invention improves the stability of the pharmaceutically active ingredient of the medicine and allows increased concentrations of the pharmaceutically active ingredients. In one aspect, the maximum temperature for long-term storage is higher than that of the known composition. In another aspect, the concentration of the composition is higher than the known composition.

Particularly, the pharmaceutical composition of the present invention is storage stable at a higher concentration and a higher temperature as compared to formulations known from the prior art. Consequently, a liquid formulation containing a typical dosage of fusion protein can be conveniently filled into a single injector commonly used. Furthermore, a frozen liquid formulation can be thawed or warmed for parenteral application in a reasonable short time. Moreover, the formulation of said fusion protein is storage stable at a non-freezing temperature, which allows the formulation to be used without a step of rehydration or thawing. Finally, the pharmaceutical composition of the present invention has an improved accessibility thanks to the low requirement for refrigeration during storage and transportation.

Furthermore, these features make the pharmaceutical composition of the present invention highly desirable for medical applications that require a fast or immediate response. For example, patients having acute serious diseases and patients that require immediate treatment. Accordingly, new medical uses of the collagen binding fusion protein is provided in the present invention.

It is a problem of the present invention to provide a liquid formulation of a collagen-binding dimeric fusion protein which retains biological activity, and has a long shelf-life.

It was not appreciated until this invention that a specific liquid formulation of a collagen-binding dimeric fusion protein in a specific buffer retains biological activity, has a long shelf-life and can be administered parenterally without lyophilization and reconstitution.

It is also a problem of the present invention to provide a pharmaceutical composition containing a collagen-binding dimeric fusion protein comprising an extracellular domain of glycoprotein VI in the prevention or treatment of for medical applications that require a fast or immediate treatment including treatment of thrombotic complications and/or bleeding complications.

It is also a problem of the present invention to provide a diagnostic method for selecting a patient that is benefiting from administration of a pharmaceutical composition containing a collagen-binding dimeric fusion protein comprising an extracellular domain of glycoprotein VI in the prevention of said bleeding complications of a certain type.

The present invention has solved the above-mentioned technical problems by providing the following:

According to a first aspect, the present invention provides a pharmaceutical composition for parenteral application, comprising
(a) more than 2.4 mg/ml of a collagen-binding dimeric fusion protein comprising an extracellular domain of glycoprotein VI, the fusion protein having an amino acid sequence of SEQ ID NO: 1, or an amino acid sequence that is at least 99% homologous to the amino acid sequence of SEQ ID NO: 1; and
(b) a pharmaceutically acceptable buffer, the buffer comprising
   (b1) a buffering component in combination with a pH adjusting agent, which is 10 mM His/HCl, and
   (b2) mannitol as a stabilizing agent in an amount of 4 percent by weight based on the total weight of the pharmaceutical composition and
   (b3) saccharose in an amount of 2.5 percent by weight based on the total weight of the pharmaceutical composition as a stabilizing agent, and
which has a pH of 7.

The present invention also provides a lyophilized composition adapted to provide a pharmaceutical composition for parenteral application as defined in any one of the preceding items after hydration.

According to a second aspect, the present invention provides a pharmaceutical composition containing a collagen-binding dimeric fusion protein comprising an extracellular domain of glycoprotein VI having an amino acid sequence of SEQ ID NO: 1, or an amino acid sequence that is at least 99% homologous to the amino acid sequence of SEQ ID NO: 1 for use in the prevention or treatment of bleeding complications in a patient by administering the pharmaceutical composition to a patient selected based on a soluble glycoprotein VI concentration in blood plasma of more than 22.8 ng/ml. The present invention also provides a kit-of-parts comprising a pharmaceutical composition of the present invention, and a diagnostic antibody directed against soluble glycoprotein VI. The present invention also provides a diagnostic method for selecting a patient benefitting from administration of a dimeric fusion protein comprising an extracellular domain of glycoprotein VI for preventing bleeding complications, which method comprises
(a) determining a soluble GPVI concentration in blood plasma of the patient; and
(b) selecting the patient if the soluble GPVI concentration is above 22.8 ng/ml.

### BRIEF DESCRIPTION OF THE FIGURES

FIG. 1 Trial profile—Consort diagram

ITT, Intention to treat population; PP, per protocol population [displayed in brackets]; CEA, Carotid endarterectomy; CAS, carotid angioplasty and stenting; BMT, best medical treatment.

FIG. 2 Primary exploratory endpoint new DWI lesion.

A) Number of new DWI lesion after revascularization procedure are numerically reduced after treatment with 40 mg Revacept (orange bar, 1.0±2.2) and 120 mg Revacept (red bar, 0.6±1.7), compared to placebo (grey bar, 1.2±2.7, p=0.529). B) Number of new DWI lesions after revascularization procedure in the subgroup of patients with >70% stenosis (according to ECST-criteria) are reduced from 1.1(±1.9) in the Placebo group (grey bar), to 0.8±1.7 after 40 mg Revacept (orange bar) and 0.5±1.1 after 120 mg Revacept (p=0.407, red bar).

FIG. 3 Influence of subgroups on frequency of patients with new DWI lesions on FU-MRI—intention to treat analysis

Predefined subgroups (Degree of ICA-stenosis, prior thrombocyte inhibition, prior statin treatment) and posthoc analysis (MES detected at Baseline, Management of ICA-stenosis) were analyzed by binary logistic regression analysis with prevalence of new DWI-lesions on FU-MRI as dependent variable and Revacept dosage 0 mg, 40 mg vs. 120 mg as covariate. Displayed is a forest plot with Odds ratio (red dot) and 95% CI (black line). OR values above 1 favor Placebo and below 1 favor Revacept treatment.

FIG. 4 Kaplan-Meier plots for cerebrovascular events or bleeding complications—intention to treat analysis

A) Time to first cerebrovascular event (any Stroke, TIA, myocardial infarction, coronary intervention) or bleeding complications derived from Cox-regression Model. Treatment with Revacept 120 mg (red line) showed a statistically significant reduction in the occurrence of combined safety and efficacy endpoint compared to placebo (grey line, p=0.047). B) Time to first cerebrovascular event (any Stroke, TIA, myocardial infarction, coronary intervention) or bleeding complication in patients with >70% stenosis (according to ECST-criteria) of the internal carotid artery. Treatment with Revacept 120 mg (red line) statistically significantly reduced the combined safety and efficacy endpoint compared to placebo (grey line, p=0.027). Grey Line (Placebo), orange line (40 mg Revacept), red line (120 mg Revacept)

FIG. 5 Influence of subgroups on the frequency of patients with occurrence of cerebrovascular event or and bleeding complication within study period—intention to treat analysis

Predefined subgroups (Degree of ICA-stenosis, prior thrombocyte inhibition, prior statin treatment) and posthoc analysis (MES detected at Baseline, Management of ICA-stenosis) were analyzed by binary logistic regression analysis with occurrence of cerebrovascular events and bleeding complications within study period as dependent variable and Revacept dosage 0 mg, 40 mg vs. 120 mg as covariate. Displayed is a forest plot with Odds ratio (red dot) and 95% CI (black line). OR values above 1 favor Placebo and below 1 favor Revacept treatment.

FIG. 6 Determination of sGPVI concentrations

The concentration of sGPVI in plasma of 30 healthy donors is determined

FIG. 7 Difference in sGPVI levels Healthy vs Patients

FIG. 8 sGPVI levels of patients with and without bleedings at visit 1

Patients with bleedings BARC1-5 show significantly higher sGPVI levels at visit 1 compared to patients without bleedings

FIG. 9 Definition of sGPVI cutoff for higher bleeding risk

FIG. 10 comparison of sGPVI levels at visit 1 and visit 2 (Placebo group)

No differences of sGPVI levels at visit 1 and visit 2 (Placebo group), no tendency of increase/decrease is found

FIG. 11 Comparison of multiplate platelet aggregation in patients with and without bleedings

Multiplate platelet aggregation is not significantly different in patients with BARC 1-5 bleedings compared to patients without bleedings

FIG. 12 Comparison of platelet counts in patients with and without bleedings

Platelet counts are not significantly different in patients with BARC 1-5 bleedings compared to patients without bleedings

### DEFINITIONS

### General

The term “about” or “approximately” preceding a figure in the present disclosure means plus or less 10% of the value of said figure, if not specifically described otherwise.

### Pharmaceutical Composition

Compositions of the present invention may be used for therapeutic or prophylactic applications. The present invention, therefore, includes a pharmaceutical composition for parenteral application, comprising a collagen-binding dimeric fusion protein comprising an extracellular domain of glycoprotein VI as disclosed herein and a pharmaceutically acceptable buffer therefor. In a related embodiment, the present invention provides a lyophilized composition adapted to provide the said pharmaceutical composition. In another related embodiment, the present invention provides a pharmaceutical composition for use in the prevention or treatment of thrombotic complications or bleeding complications. In another related embodiment, the present invention provides a kit-of-parts comprising the pharmaceutical composition of the present invention and a diagnostic antibody.

### Parenteral Application

The pharmaceutical composition of the present invention is suitable for parenteral application. For example, the pharmaceutical composition of the present invention can be administered in a parenteral route. Parenteral administration can be performed by injection, that is, using a needle, usually a hypodermic needle, and a syringe, or by the insertion of an indwelling catheter. The term injection encompasses intravenous (IV), intramuscular (IM), subcutaneous (SC) and intradermal (ID) administration. Other routes of parenteral administration may also include, intraarterial, intraperitoneal, or intranasal administration. Suppositories or transdermal patches can also be employed.

When parenteral application is needed or desired, particularly suitable admixtures for the compositions, fusion proteins and proteins are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compositions, proteins or polypeptides can also be administered by transdermal pumps or patches. Pharmaceutical admixtures suitable for use in the present invention are well-known to person skilled in the art and are described, for example, in Remington's Pharmaceutical Sciences (18th Ed., Mack Pub. Co., Easton, PA).

### Homologous Sequences

“Homologous sequences” mean nucleotide or amino acid sequences having a percentage of nucleotides or amino acids identical at corresponding positions which is higher than in purely random alignments. They are considered as homologous when they show a minimum of homology (or sequence identity) defined as the percentage of identical nucleotides or amino acids found at each position compared to the total nucleotides, after the sequences have been optimally aligned taking into account additions or deletions (like gaps) in one of the two sequences to be compared. Methods of alignment of sequences are based on local homology algorithms which have been computerized and are available as for example (but not limited to) Clustal®, (Intelligenetics, Mountain Views, California), or GAP®, BESTFIT®, FASTA® and TFASTA® (Wisconsin Genetics Software Package, Genetics Computer Group Madison, Wisconsin, USA) or Boxshade®.

### Collagen-Binding Dimeric Fusion Protein

The GPVI-Fc fusion protein of the present invention is a collagen-binding dimeric fusion protein. Specifically, said collagen-binding dimeric fusion protein comprises an extracellular domain of GPVI. More specifically, said GPVI-Fc has an amino acid sequence of SEQ ID NO: 1, or an amino acid sequence that is at least 99% homologous to the amino acid sequence of SEQ ID NO: 1.

(SEQ ID NO: 1)
MSPSPTALFCLGLCLGRVPAQSGPLPKPSLQALPSSLVPLEKPVTLRCQG
PPGVDLYRLEKLSSSRYQDQAVLFIPAMKRSLAGRYRCSYQNGSLWSLPS
DQLELVATGVFAKPSLSAQPGPAVSSGGDVTLQCQTRYGFDQFALYKEGD
PAPYKNPERWYRASFPIITVTAAHSGTYRCYSFSSRDPYLWSAPSDPLEL
VVTGTSVTPSRLPTEPPSSVAEFSEATAELTVSFTNKVFTTETSRSITTS
PKESDSPAGPARQYYTKGNGGRESKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKT
TPPVLDSDGSFFLYSKLTVDKSRWQQGNVFSCSVMHEALHNHYTQKSLSL
SPGK

### Dimeric Fusion Protein

The term “fusion proteins” or “chimeric proteins” refers to proteins created through the joining of two or more genes that originally coded for separate proteins. For example, Revacept® (GPVI-Fc) is a known fusion protein that consists of the Fc part of human IgG1, a short hinge region derived of the same protein, a specific linker sequence, and the extracellular part of human GPVI.

The term “dimeric”, refers to dimerized protein or protein dimer. A protein dimer is a macromolecular complex formed by two protein monomers, or single proteins, wherein the monomers are bound covalently or non-covalently. For example, the protein dimer may be bound covalently via one or more disulfide bridges. The one or more disulfide bridges may be reversibly formed or broken in response to the environmental redox potentials. Specifically, a dimeric fusion protein is a fusion protein dimer or dimerized fusion proteins. For example, GPVI-Fc is present as covalently linked dimer.

### Collagen Binding

The term “collagen binding” refers to the ability to bind specifically to collagen. For example, a collagen binding protein specifically binds to collagen in the subendothelial matrix. Specifically, a collagen binding dimeric fusion protein is a dimeric fusion protein that binds specifically to collagen.

As used herein the term “binds specifically to”, “specifically binds to”, is “specific to/for” or “specifically recognizes”, or the like, refers to measurable and reproducible interactions such as binding between a target and an antibody or antibody fragment, which is determinative of the presence of the target in the presence of a heterogeneous population of molecules including biological molecules. For example, an antibody or antibody fragment that specifically binds to a target (which can be an antigen or an epitope of an antigen) is an antibody or antibody fragment that binds this target with greater affinity, avidity, more readily, and/or with greater duration than it binds to other targets. In certain embodiments, an antibody or antibody fragment specifically binds to an epitope on a protein that is conserved among the protein from different species. In another embodiment, specific binding can include, but does not require exclusive binding. The antibodies or antibody fragments disclosed herein specifically bind to human GPVI. Preferably, the disclosed antibodies or antibody fragments specific for human GPVI specifically bind to GPVI of another species, such as GPVI from mouse, rat and/or cynomolgus monkey. Even more preferred, the antibodies or antibody fragments disclosed herein are specific for human GPVI, cynomolgus monkey GPVI, mouse GPVI and rat GPVI. Methods for determining whether two molecules specifically bind are well known in the art and include, for example, a standard ELISA assay. The scoring may be carried out by standard color development (e.g. secondary antibody with horseradish peroxide and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative can be more than 5-fold. Typically, determination of binding specificity is performed by using not a single reference antigen, but a set of three to five unrelated antigens, such as milk powder, BSA, transferrin or the like.

As used herein, the term “affinity” refers to the strength of interaction between the polypeptide and its target at a single site. Within each site, the binding region of the polypeptide interacts through weak non-covalent forces with its target at numerous sites; the more interactions, the stronger the affinity.

For example, the fusion protein of the present invention is a recombinant protein which is a fusion protein between the GPVI extracellular domain and a human Ig Fc domain. Said fusion protein forms a dimer. Said dimeric fusion protein competes with platelet GPVI for specifically binding collagen.

The term “antibody” as used herein refers to a protein comprising at least two heavy (H) chains and two light (L) chains inter-connected by disulfide bonds which interacts with an antigen. Each heavy chain (HC) is comprised of a heavy chain variable region (abbreviated herein as VH) and a heavy chain constant region. The heavy chain constant region is comprised of three domains, CH1, CH2 and CH3. Each light chain (LC) is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., IgG1, IgG2, IgG3, IgG4, IgA1 and IgA2) or subclass. Both the light and heavy chains are divided into regions of structural and functional homology.

The phrase “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a Fab′ fragment, a monovalent fragment consisting of the VL, VH, CL and CH1 domains and a N-terminal portion of the hinge region of an immunoglobulin; a F(ab)₂ fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1126-1136). Antibody fragments can be grafted into scaffolds based on polypeptides such as Fibronectin type III (Fn3) (see U.S. Pat. No. 6,703, 199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870). As used herein, the term “hinge region” includes the region of an antibody heavy chain that joins the CH1 domain to the CH2 domain. Hinge regions can be subdivided into three distinct domains: upper, middle, and lower hinge domains (Roux et ah, J. Immunol. 1998 161:4083).

The term “CH1 domain” refers to the heavy chain constant domain of an antibody linking the variable domain to the hinge region. The term “CH1 domain” includes wildtype CH1 domains and one of its natural occurring allotypes as well as variants thereof.

A “human antibody” or “human antibody fragment”, as used herein, includes antibodies and antibody fragments having variable regions in which both the framework and CDR regions are derived from sequences of human origin. Furthermore, if the antibody contains a constant region, the constant region also is derived from such sequences. Human origin includes, e.g., human germline sequences, or mutated versions of human germline sequences or antibody containing consensus framework sequences derived from human framework sequences analysis, for example, as described in Knappik et al., (2000) J Mol Biol 296:57-86). The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g., Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948. Human antibodies can also be isolated from synthetic libraries or from transgenic mice (e.g. xenomouse) provided the respective system yield in antibodies having variable regions in which both the framework and CDR regions are derived from sequences of human origin.

### Extracellular Domain of GPVI

The term “GPVI” refers to a protein known as Glycoprotein VI.

Human GPVI-1A (1-339) has the amino acid sequence of (Uniprot: Q9HCN6-1, haplotype ‘a’)

MSPSPTALFCLGLCLGRVPAQSGPLPKPSLQALPSSLVPLEKPVTLRCQG
PPGVDLYRLEKLSSSRYQDQAVLFIPAMKRSLAGRYRCSYQNGSLWSLPS
DQLELVATGVFAKPSLSAQPGPAVSSGGDVTLQCQTRYGFDQFALYKEGD
PAPYKNPERWYRASFPIITVTAAHSGTYRCYSFSSRDPYLWSAPSDPLEL
VVTGTSVTPSRLPTEPPSSVAEFSEATAELTVSFTNKVFTTETSRSITTS
PKESDSPAGPARQYYTKGNLVRICLGAVILIILAGFLAEDWHSRRKRLRH
RGRAVQRPLPPLPPLPLTRKSNGGQDGGRQDVHSRGLCS

Signal peptide/Extracellular Domain/Transmembrane and cytoplasmic domains Cynomolgus monkey GPVI (1-318) has the amino acid sequence of (UniProt BOI1T7):

(SEQ ID NO: 2)
MSPSPTTLFCLGLCLGHVPAQRGPLPKPSLQALPSSLVPLEKPVTLRCQG
PPGVDLYRLEKLSSSRYQDQAVLFIPAMKRHLAGRYRCSYQNGSLWSPPS
DQLELVATGVFAKPSLSAQPGPAVSSGGDVTLQCQTRYGFDQFALYKEGD
PAPYKNPERWYRASFPIITVTAAHSGTYRCYSFSSGDPYLWSAPSDPLEL
MVTEFSEATTELTVSLTNKVFTTETSRSITASPKEPGSPAGPARQYYTKG
NLVRICLGAVILILLAGFLAEDWHSRRKRLRHRVRAVQRPLPPLPPTRKS
HGDQDGGRPDVHSRGLCS

Murine GPVI (1-313) has the amino acid sequence of (UniProt POC191):

(SEQ ID NO: 3)
MSPASPTFFCIGLCVLQVIQTQSGPLPKPSLQAQPSSLVPLGQSVILRCQ
GPPDVDLYRLEKLKPEKYEDQDFLFIPTMERSNAGRYRCSYQNGSHWSLP
SDQLELIATGVYAKPSLSAHPSSAVPQGRDVTLKCQSPYSFDEFVLYKEG
DTGSYKRPEKWYRANFPIITVTAAHSGTYRCYSFSSSSPYLWSAPSDPLV
LVVTGLSATPSQVPTEESFPVTESSRRPSILPTNKISTTEKPMNITASPE
GLSPPFGFAHQHYAKGNLVRICLGATIIIILLGLLAEDWHSRKKCLQHRM
RALQRPLPPLPLA

The extracellular domain of human GPVI-1A (Position 21-269) has the amino acid sequence of (Uniprot: Q9HCN6-1, haplotype ‘a’):

(SEQ ID NO: 4)
QSGPLPKPSLQALPSSLVPLEKPVTLRCQGPPGVDLYRLEKLSSSRYQDQ
AVLFIPAMKRSLAGRYRCSYQNGSLWSLPSDQLELVATGVFAKPSLSAQP
GPAVSSGGDVTLQCQTRYGFDQFALYKEGDPAPYKNPERWYRASFPIITV
TAAHSGTYRCYSFSSRDPYLWSAPSDPLELVVTGTSVTPSRLPTEPPSSV
AEFSEATAELTVSFTNKVFTTETSRSITTSPKESDSPAGPARQYYTKGN

The extracellular domain of human GPVI-1B (Position 21-269) has the amino acid sequence of (Uniprot: Q9HCN6-1, haplotype ‘b’):

(SEQ ID NO: 5)
QSGPLPKPSLQALPSSLVPLEKPVTLRCQGPPGVDLYRLEKLSSSRYQDQ
AVLFIPAMKRSLAGRYRCSYQNGSLWSLPSDQLELVATGVFAKPSLSAQP
GPAVSSGGDVTLQCQTRYGFDQFALYKEGDPAPYKNPERWYRASFPIITV
TAAHSGTYRCYSFSSRDPYLWSAPSDPLELVVTGTSVTPSRLPTEPPSPV
AEFSEATAELTVSFTNEVFTTETSRSITASPKESDSPAGPARQYYTKGN

The extracellular domain of human GPVI-2A (Position 21-251) has the amino acid sequence of (Uniprot: Q9HCN6-2, haplotype ‘a’):

(SEQ ID NO: 6)
QSGPLPKPSLQALPSSLVPLEKPVTLRCQGPPGVDLYRLEKLSSSRYQDQ
AVLFIPAMKRSLAGRYRCSYQNGSLWSLPSDQLELVATGVFAKPSLSAQP
GPAVSSGGDVTLQCQTRYGFDQFALYKEGDPAPYKNPERWYRASFPIITV
TAAHSGTYRCYSFSSRDPYLWSAPSDPLELVVTEFSEATAELTVSFTNKV
FTTETSRSITTSPKESDSPAGPARQYYTKGN

The terms “GPVI-1” and “GPVI-2” refer to the published isotypes of GPVI. The suffixes “A” and “B” refer to the described high frequency allele “a” (comprising amino acids S219, K237, T249) and low frequency allele “b” (comprising amino acids Pro219, Glu237, Ala249), respectively (Joutsi-Korhonen et al., 2003).

The extracellular domain of human GPVI is composed of two lg-like C2-type domains, namely the D1 domain and the D2 domain, linked by a hinge-interdomain. The D1 domain comprises amino acid residues 21 to 109 of human GPVI-1A (1-339). The D2 domain comprises amino acid residues 114 to 207 of Human GPVI-1A (1-339)

The extracellular domain of cynomolgus monkey GPVI (Position 21-249) has the amino acid sequence of (Uniprot: BOI1T7):

(SEQ ID NO: 7)
QRGPLPKPSLQALPSSLVPLEKPVTLRCQGPPGVDLYRLEKLSSSRYQDQ
AVLFIPAMKRHLAGRYRCSYQNGSLWSPPSDQLELVATGVFAKPSLSAQP
GPAVSSGGDVTLQCQTRYGFDQFALYKEGDPAPYKNPERWYRASFPIITV
TAAHSGTYRCYSFSSGDPYLWSAPSDPLELMVTEFSEATTELTVSLTNKV
FTTETSRSITASPKEPGSPAGPARQYYTK

The extracellular domain of mouse GPVI (Position 21-266) has the amino acid sequence of (Uniprot: POC191):

(SEQ ID NO: 8)
QSGPLPKPSLQAQPSSLVPLGQSVILRCQGPPDVDLYRLEKLKPEKYEDQ
DFLFIPTMERSNAGRYRCSYQNGSHWSLPSDQLELIATGVYAKPSLSAHP
SSAVPQGRDVTLKCQSPYSFDEFVLYKEGDTGSYKRPEKWYRANFPIITV
TAAHSGTYRCYSFSSSSPYLWSAPSDPLVLVVTGLSATPSQVPTEESFPV
TESSRRPSILPTNKISTTEKPMNITASPEGLSPPFGFAHQHYAKGN

The extracellular domain of rat GPVI (Position 21-269) has the amino acid sequence of (Uniprot: XP_008757241.2):

(SEQ ID NO: 9)
QHGPLPKPSLQAQPSSLVPLGHPVTLRCLGPSDADLYRLEKVKPGKLIFI
DQDFLFIPIMEINNAGRYRCSYQNESHWSLPSDQLELIATGVYSKPSLSA
HPSSAIPPGRDVTLKCQSQYSFDEFVLYKEGDTRPYKRPEKWYRANFPVI
TVTAAHSGTYRCYSFSSSSPYLWSAPSDPLVVVVTGPSATPSQVPTEVPS
PMTEASRRPSMLLTNKISTTEKPMNITVSPEGPSPPFGFAHQHYAKGN

The human IgG1-Fc domain (K105-K330) used for the generation of GPVI-Fc fusion protein has the amino acid sequence of:

(SEQ ID NO: 10)
DIKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQ
GNVFSCSVMHEALHNHYTQKSLSLSPGK

### Pharmaceutically Acceptable Buffer

The term “pharmaceutically acceptable” means a non-toxic material that does not decrease the effectiveness of the biological activity of the active ingredients. The term “pharmaceutically acceptable buffer” refers to a buffer that is not biologically or otherwise undesirable, and that can be administered to a person or an animal without causing any undesirable biological effects or interacting in a deleterious manner with other products or components contained in the vesicles. Pharmaceutically acceptable buffers are used to control the pH value of a pharmaceutical formulation at a desired predetermined pH value. Pharmaceutically acceptable buffers, diluents, carriers and excipients are well-known in the art (see Remington's Pharmaceutical Sciences, 18th edition, A. R Gennaro, Ed., Mack Publishing Company (1990) and Handbook of Pharmaceutical Excipients, 3rd edition, A. Kibbe, Ed., Pharmaceutical Press (2000)).

### Storage Stable

The term “storage stable” or “storage stability” refers to stability or stable during the storage of a pharmaceutical composition. The term “stability” or “stable” as used herein, refers to stability or stable in terms of specific bioactivity and/or changes in secondary structure from the native proteins. A pharmaceutical composition is storage stable if it is stable at a predetermined range of temperature for a certain amount of time.

### Unit Dose

The term “unit dose”, as used in the present disclosure, is a discrete amount of the pharmaceutical composition comprising a predetermined amount of API. The amount of API is generally equal to the dosage of API which would be administered to a patient or a convenient fraction of such a dosage such as, for example, one-half or one-third of such a dosage. For example, the API of the pharmaceutical composition according to the present invention is the collagen-binding dimeric fusion protein comprising an extracellular domain of glycoprotein VI, said fusion protein having a certain amino acid sequence as defined in the present disclosure.

### Thrombotic Complications

The term “thrombotic complications” is an art-recognized term, which should be understood to include medical complications arising from the formation of thrombosis in a subject. Thrombosis occurring in veins and arteries are referred to as venous thrombosis and arterial thrombosis respectively. Examples of thrombotic complication of different origins include a thrombotic complication of an atherosclerotic disease, a thrombotic complication of an intervention of an atherosclerotic disease, a thrombotic complication associated with surgical or mechanical damage, thrombotic complications associated with thrombolytic therapy, thrombotic complications associated with coronary and other angioplasty, thrombotic complications associated with coronary artery bypass procedures. Serious thrombotic complications include stroke, heart attack or myocardial infarction (MI), and serious breathing problems.

Thrombotic complications related to GPVI may include, but are not limited to thrombotic or vascular (e.g. cardiovascular) disorders or conditions, such as, for example, arterial thrombosis including atherothrombosis, ischemic events, acute coronary artery syndrome, myocardial infarction (heart attack), acute cerebrovascular ischemia (stroke), limb ischemia, percutaneous coronary intervention, stenting thrombosis, bypass thrombosis and occlusion, ischemic, restenosis, ischemia, (acute and chronic), disorders of the aorta and its branches (such as aortic aneurysm, thrombosis), peripheral artery disorders, venous thrombosis including cerebral and sinus venous thrombosis, acute phlebitis and pulmonary embolism, cancer-associated thrombosis (Trousseau syndrome), immunogenic thrombotic thrombocytopenia (ITP) including vaccine induced ITP, inflammatory thrombosis and thrombosis associated to infection.

### Acute Cardiovascular Event

The term “acute cardiovascular event” refers to all events which suddenly appear, i.e. without previous clinical signs or symptoms, and which severely affect the diastolic or systolic blood flow rate. Histopathologically, the acute cardiovascular event referred to herein shall be accompanied by a sudden ischemia of heart muscle cells accompanied by severe necrosis of said cells, the brain with nerve cells and accompanying cells, muscles, kidney or other organs depending on sufficient blood supply endangered by thrombotic vessel occlusion. Optionally, the subject suffering from an acute cardiovascular event will also suffer from typical symptoms such as chest, epigastric, arm, wrist or jaw discomfort or pain whereby, in particular, the chest pain may radiate to the arm, back or shoulder. Further symptoms of an acute cardiovascular event may be unexplained nausea or vomiting, persistent shortness of breath, weakness, dizziness, lightheadedness or syncope as well as any combinations thereof. Optionally, the acute cardiovascular event referred to herein is an acute coronary syndrome (ACS), i.e. either an unstable angina pectoris (UAP) or myocardial infarction (MI). Preferably, the acute cardiovascular event is MI including ST-elevated MI and non-ST-elevated MI. Further details on the definitions, symptoms and clinical signs such as electrocardiograms, are found in Joint European Society of Cardiology/American Society of Cardiology, 2000, J American College of Cardiology, Vol. 36, No. 3:959-969. Symptoms may be classified according to the New York Heart Association classification system. Accordingly, patients can be divided into individuals showing no clinical symptoms and those with symptoms (e.g. dyspnea). Examples of acute cardiovascular event include acute coronary syndrome (ACS), heart attack or myocardial infarction (MI), unstable angina pectoris (UAP), acute decompensated heart failure (ADHF), myocardial ischemia, chronic stable angina pectoris, unstable angina pectoris, angioplasty, stroke, transient ischemic attack, claudication(s), vascular occlusion(s), and peripheral artery disease(s). Preferably, the cardiovascular event as referred to in the present invention is selected from the group consisting of stroke, myocardial infarction (MI) and peripheral artery disease.

### Degree of Carotid Stenosis

Carotid artery stenosis is a narrowing or constriction of any part of the carotid arteries. Carotid artery stenosis is usually diagnosed by color flow duplex ultrasound scan of the carotid arteries in the neck. The term “degree of carotid stenosis” or stenosis degree refers to a parameter used in the choice of therapeutic options for carotid artery stenosis. Degree of carotid stenosis is defined differently by the North American Symptomatic Carotid Endarterectomy Trial (NASCET) and European Carotid Surgery Trial (ECST). (Randomised trial of endarterectomy for recently symptomatic carotid stenosis: final results of the MRC European Carotid Surgery Trial (ECST). Lancet. 1998; 351 (9113):1379-87)

NASCET criteria was established by angiographic calculation of ICA stenosis percentage using the following formula: % ICA stenosis=(1−[narrowest ICA diameter/diameter normal distal cervical ICA])×100

ECST criteria was established by angiographic calculation of ICA stenosis percentage using the following formula: % ICA stenosis=(1−[diameter of the most stenotic part/estimated original diameter at the site of the stenosis])×100

NASCET criteria and ECST criteria are both accepted as parameters in the choice of therapeutic options. For example, NASCET demonstrated a conclusive benefit for carotid endarterectomy in patients with symptomatic 70-99% ICA stenosis according to NASCET criteria. ECST also demonstrated benefits for carotid endarterectomy in patients with symptomatic higher than 80% ICA stenosis according to ECST criteria.

### Antiplatelet Therapy

The term “antiplatelet therapy” refers to a treatment with one or more of antiplatelet drugs to decrease the ability of blood clots to form by interfering with the platelet activation process in primary hemostasis. The term antiplatelet drug as used herein refers to antiaggregant, platelet agglutination inhibitor, or platelet aggregation inhibitor, are medicaments that decreases platelet aggregation and inhibit thrombus formation. Examples of antiplatelet drugs include Aspirin, Triflusal, and P2Y₁₂ inhibitors including Clopidogrel, Prasugrel, Ticagrelor. Example of antiplatelet therapy includes low dose aspirin.

The term “standard therapy” or “standard treatment” is an art-recognized term, which is also referred to as “best practice”, “standard medical care”, and “standard of care”. Standard therapy should be understood to include treatments that is currently in wide use and approved by health authorities, which is considered to be the most effective and/or efficient therapy for a specific disease or condition. A “standard antiplatelet therapy” is a standard therapy for antiplatelet treatment. So far, dual antiplatelet therapy which typically combines acetylsalicylic acid (ASA) with an ADP receptor antagonist such as clopidogrel is the standard therapy for patients with acute vascular lesions treated by coronary stenting, and its major limitation is increased bleeding risk. For example, dual antiplatelet therapy with aspirin and a P2Y₁₂ inhibitor is standard antiplatelet therapy after acute coronary syndromes.

### Revascularization Procedures

The term “revascularization procedure”, as used herein, relates to a procedure aimed at the restoration of perfusion to a body part or body organ that has suffered from ischemia. Revascularization procedures include, without limitation, angioplasty or percutaneous transluminal angioplasty (PTA), such as coronary angioplasty, stenting, such as carotid artery stenting (CAS), a combination of angioplasty and stenting, such as percutaneous coronary intervention (PCI), vascular bypass, such as coronary artery bypass grafting (CABG) or peripheral artery bypass, endarterectomy, such as carotid endarterectomy (CEA), and atherectomy.

### Thrombolysis and Thrombectomy

The term “thrombolysis” as used herein refers to the pharmacological breakdown of a blood clot, regardless of the particular drug or pharmacological treatment used. Thrombolysis may involve the injection of a thrombolytic drug through an intravenous (IV) line or through a long catheter that delivers the drug directly to the site of the blockage.

The term “thrombectomy” as used herein refers to any surgical removal or breakdown of a clot, regardless of the method used. Mechanical thrombectomy devices for thrombectomy include coil retrievers, aspiration devices, and stent retrievers. In a preferred embodiment, said thrombectomy device is stent retriever.

Thrombolysis and thrombectomy and may be combined for practical reasons. Thrombolytic treatment is usually initiated before thrombectomy can be performed, but the steps of any method disclosed herein do not have to be performed in the exact order disclosed, unless explicitly stated.

### Bleeding Complications

The term “bleeding complications” is an art-recognized term, which should be understood to include bleeding events such as cerebral bleeding (such as haemorrhagic stroke or intracranial haemorrhage) or gastrointestinal bleeding.

Bleeding complications can be perioperative or postoperative. The term “perioperative bleeding” includes any bleedings occurring in the time period surrounding a patient's surgical or operative procedure. Before the surgery or operative procedure starts the perioperative bleeding may include bleedings associated with e.g. epidural anesthesia or other invasive procedures. Depending on the exact circumstances, this may include a time period before the surgery/operation such as e.g. 12 hours before, 5 hours before, 1 hour before or 30 minutes before surgery, but also includes intraoperative (occurring during the surgery/operation) and postoperative bleedings (occurring after surgery). The term “postoperative bleedings” includes any bleedings after surgery or operation. In the present context it is intended to cover the time period up to 48 hours after surgery, such as e.g. 12 hours after surgery, 6 hours after surgery, 3 hours after surgery, 1 hour after surgery, or less.

### Soluble Glycoprotein VI (sGPVI)

The term “soluble glycoprotein VI” or “soluble GPVI” or “sGPVI” is GPVI as cleaved or shed from the platelet surface. sGPVI is a marker of platelet activation in thrombotic conditions or for bleeding complications in patients. The concentration of sGPVI in blood plasma can be detected, for example, by a sandwich ELISA assay.

### Percutaneous Intervention

The term “percutaneous intervention” is an art-recognized term, which should be understood to include any surgical procedure or method where access to inner organs or other tissue is done via needle-puncture of the skin, where inner organs or tissue are not exposed. For example, said percutaneous intervention is percutaneous coronary intervention (PCI).

### Embodiments

The present invention relates to a pharmaceutical composition for parenteral application, comprising
(a) more than 2.4 mg/ml of a specific collagen-binding dimeric fusion protein comprising an extracellular domain of glycoprotein VI, the fusion protein having an amino acid sequence of SEQ ID NO: 1, or an amino acid sequence that is at least 99% homologous to the amino acid sequence of SEQ ID NO: 1; and
(b) a specific pharmaceutically acceptable buffer, the buffer comprising (b1) a buffering component in combination with a pH adjusting agent.

### Composition of Fusion Protein and Amount Thereof

In a preferred embodiment, said pharmaceutical composition comprises (a) a collagen-binding dimeric fusion protein in an amount of at least 2.5 mg/ml, at least 3.0 mg/ml, at least 3.5 mg/ml, at least 4.0 mg/ml, at least 4.5 mg/ml, at least 5.0 mg/ml, at least 5.5 mg/ml, or, at least 6.0 mg/ml. Preferably, in an amount of at least 4.0 mg/ml, at least 5.0 mg/ml or at least 6.0 mg/ml. More preferably, in an amount of at least 5.0 mg/ml. For example, said pharmaceutical composition comprises (a) a collagen-binding dimeric fusion protein having an amino acid sequence of SEQ ID NO: 1 in an amount in the range of more than 2.4 up to 10 mg/ml, more preferably 2.5 to 9 mg/ml, still more preferably 3.0 to 8.5 mg/ml, still more preferably 4.0 to 8 mg/ml, and a pharmaceutically acceptable buffer. For example, said pharmaceutical composition comprises a collagen-binding dimeric fusion protein having an amino acid sequence of SEQ ID NO: 1 in an amount of 4.0 mg/ml or more, and a pharmaceutically acceptable buffer. For example, said pharmaceutical composition comprises GPVI-Fc (Revacept®) in an amount of 5.0 mg/ml or more, and a pharmaceutically acceptable buffer.

The term “buffering component” in a buffer refers to a compound that is capable of providing pH buffering capacity. Such compounds are typically a weak acid or its conjugate base (usually in the salt form), a weak base or its conjugate acid (usually in the salt form), a twitter ionic compound, or mixtures thereof.

The term “pH adjusting agent” in a buffer refers to a compound that is used to adjust the pH value of a buffer to a predetermined value as needed. Such compound is typically a strong acid or a strong base, such as HCl, H₂SO₄, KOH, or NaOH. Such compound may also be a weak acid or a weak base, such as acetic acid or ammonia solution. Such compound may also be a conjugated acid or base in a buffer system. For example, for a phosphate buffer system, sodium phosphate monobasic is a conjugated acid, and sodium phosphate dibasic is a conjugated base.

Pharmaceutically acceptable buffers used in a pharmaceutical composition include acetate buffers (e.g. sodium acetate), citrate buffers (e.g. sodium citrate), and phosphate buffers (e.g. sodium phosphate), succinate buffers (e.g. sodium succinate) and amino acid buffers (e.g. Arginine, Asparagine, Glutamic acid, Glutamine, Glycine, Histidine, Isoleucine, Leucine, Lysine, Methionine, Phenylalanine, Serine, Threonine, Valine, Cysteic acid, N-Glycylglycinen, Ornithine), Good's buffers (e.g. ADA, MES, Bis-tris methane (BTM), Bis-tris propane (BTP), PIPES, ACES, MOPSO, Cholamine chloride, MOPS, BES, TES, HEPES, DIPSO, MOBS, Acetamidoglycine, TAPSO, TEA, POPSO, HEPPSO, EPS, HEPPS (EPPS), Tricine, Tris, Glycinamide, Glycylglycine (Gly-Gly), HEPBS, Bicine, TAPS (N-tris(Hydroxymethyl)methyl-4-aminopropanesulfonic acid), TPBS (N-tris(Hydroxymethyl)methyl-4-aminobutanesulfonic acid), AMP, AMPD (2-Amino-2-methyl-1,3-propanediol, Ammediol), AMPSO, AMPB, CHES, CAPSO, CAPS, CABS). Other commonly known pharmaceutically acceptable buffers include bacteriostatic water for injection (BWFI), phosphate-buffered saline (PBS), Ringer's solution and Intravenous sugar solution (dextrose solution).

### Buffering Component

According to the present invention, the pharmaceutical composition comprises a buffering component in combination with a pH adjusting agent. The (b1) buffering component in combination with a pH adjusting agent is His/HCl.

In one embodiment, the pharmaceutical composition of the present invention comprises (a) more than 2.4 mg/ml of a collagen-binding dimeric fusion protein as stated above, and (b1) about 10 mM His/HCl buffer.

### Stabilizing Agent

Said (b) buffer of the pharmaceutical composition comprises mannitol in an amount of about 4% by weight based on the total weight of the pharmaceutical composition.

The (b3) disaccharide is sucrose in an amount of about 2.5% by weight based on the total weight of the pharmaceutical composition.

The (b) buffer further comprises a combination of a (b2) sugar alcohol and (b3) a non-reducing disaccharide.

Accordingly, the (b) buffer comprises (b1) His/HCl, (b2) a sugar alcohol and (b3) a disaccharide. Specifically, the (b) buffer comprises (b1) His/HCl, (b2) mannitol, and (b3) sucrose.

### Detergent

In a further preferred embodiment, wherein the (b) buffer further comprises (b4) a detergent. For example, the (b) buffer comprises (b1) Tris/acetic acid, (b2) mannitol, (b3) sucrose, and (b4) a detergent, such as Tween 20, Tween 40 or Tween 80. For example, the (b) buffer comprises (b1) Tris/acetic acid, (b2) sorbitol, (b3) sucrose, and (b4) a detergent, such as Tween 20, Tween 40 or Tween 80.

Preferably, for example, the (b) buffer comprises (b1) His/HCl, (b2) mannitol, (b3) sucrose, and (b4) a detergent, such as Tween 20, Tween 40 or Tween 80.

### pH Value

The pharmaceutical composition of the present invention has a pH value of 7.0.

The term “about” as used herein for pH values refers to a range with a variation of 2 in the last significant figure, including the boundary value. For example, “about 7.40” refers to any value between and including 7.38 and 7.42.

### Storage Stability

In a preferred embodiment, the pharmaceutical composition for parenteral application is storage stable at a temperature of 8° C. for at least 6 months. Preferably, for at least 12 months. More preferably, for at least 24 months. For example, the pharmaceutical composition for parenteral application is storage stable at a temperature of 8° C. for at least 6 months, at least 7 months, at least 8 months, at least 9 months, at least 10 months, at least 11 months, at least 12 months, at least 18 months, at least 24 months, at least 32 months, at least 36 months. Preferably, for example, the pharmaceutical composition for parenteral application is storage stable at a temperature of 8° C. for at least 24 months. Preferably, for example, the pharmaceutical composition for parenteral application is storage stable at a temperature of 20° C. for at least 12 months.

### Aqueous Dispersion

In a preferred embodiment, the pharmaceutical composition for parenteral application is an aqueous dispersion. The term “aqueous dispersion” refers to a dispersion in an aqueous media. A dispersion is a system in which distributed particles of one material are dispersed in a continuous phase of another material. An example of said distributed particles of one material in the present disclosure is protein or clusters of protein. Said continuous phase of another material in the present disclosure is an aqueous media. An aqueous media as used herein refers to water and solution in water comprising at least one solute.

### Package in Unit Dose

In a preferred embodiment, the pharmaceutical composition for parenteral application is packaged in a unit dose perfusion syringe containing at most 50 ml of the composition. A unit-dose perfusion syringe is a pre-filled perfusion syringe containing a predetermined unit dose of the pharmaceutical composition. The term “perfusion syringe” in the present disclosure refers to a syringe suitable for perfusion application, such as injection of a medicament into blood vessel.

### Other Excipient in the Composition

In one embodiment, the pharmaceutical composition of the present invention further comprises one or more pharmaceutically acceptable excipients that are suitable for parenteral applications. Said excipients include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

### Composition Suitable For Drying

In one embodiment, the pharmaceutical composition of the present invention is suitable to be dried. Methods of drying that is suitable for the pharmaceutical composition of the present invention include, but are not limited to air drying with unheated forced air, indirect or contact drying via heating through a hot wall (e.g. drum drying, vacuum drying, rotary vacuum drying), dielectric drying (e.g. microwave assisted drying), freeze drying or lyophilization, supercritical drying via superheated steam, or a combination thereof. Preferably, the method of drying is freeze drying or lyophilization. In one embodiment, the buffer to be used in the present invention is suitable for a solution to be dried. In a preferred embodiment, the buffer to be used in the present invention is suitable for freeze drying or lyophilization.

### Lyophilized Composition

The present invention also relates to a lyophilized composition adapted to provide a pharmaceutical composition for parenteral application of the present invention after hydration. The term “lyophilized composition” in the present disclosure refers to a pharmaceutical composition obtained by lyophilization or freeze-drying of an aqueous mixture. The term “freeze-drying”, “lyophilized”, “lyophilisation”, or “lyophilization” is meant to encompass a cryodesiccation, which is a dehydration process wherein the item being lyophilized is freeze-dried. The freeze-drying process can be performed in a manifold freeze-dryer, a rotary freeze-dryer and a tray style freeze-dryer. A lyophilized composition can be adapted to provide a pharmaceutical composition after hydration. The term “hydration” as used herein means “rehydration” or “reconstitution”, which refers to the addition of a suitable solvent to the lyophilized composition. The solvent may be selected from water, blood, serum, plasma, media (for example cell media) or a suitable buffer. The physiological fluid (for example, blood, serum or plasma) may be taken from the subject to whom the composition is being administered to improve compatibility. The solvent may optionally include one or more additional components, such as nutrients, growth factors, drugs, cells, etc.

### Storage Stability of Lyophilized Composition

In a preferred embodiment, the lyophilized composition is storage stable at 25° C. for at least 6 months. For example, the lyophilized composition is storage stable at a temperature of 25° C. for at least 6 months, at least 7 months, at least 8 months, at least 9 months, at least 10 months, at least 11 months, at least 12 months, at least 18 months, at least 24 months, at least 32 months, at least 36 months.

In a preferred embodiment, the lyophilized composition is storage stable at 25° C. for at least 6 months. Preferably, for at least 12 months. More preferably, for at least 24 months. For example, the pharmaceutical composition for parenteral application is storage stable at a temperature of 25° C. for at least 6 months, at least 7 months, at least 8 months, at least 9 months, at least 10 months, at least 11 months, at least 12 months, at least 18 months, at least 24 months, at least 32 months, at least 36 months. Preferably, for example, the pharmaceutical composition for parenteral application is storage stable at a temperature of 25° C. for at least 24 months. Preferably, for example, the pharmaceutical composition for parenteral application is storage stable at a temperature of −20° C. for at least 24 months.

### Medical Use of Fusion Protein of SEQ #1

In a preferred embodiment, the fusion protein of the pharmaceutical composition for use in the prevention or treatment of thrombotic complications according to the present invention is a collagen-binding dimeric fusion protein having an amino acid sequence of SEQ ID NO: 1, or an amino acid sequence that is at least 99% homologous to the amino acid sequence of SEQ ID NO: 1.

### Medical Use For Thrombotic Complications

In a particular embodiment, the pharmaceutical composition for use in the prevention or treatment of thrombotic complications according to the present invention is a pharmaceutical composition as defined above according to the present invention. Particularly, said pharmaceutical composition for use in the prevention or treatment of thrombotic complications according to the present invention is a pharmaceutical composition for parenteral application, comprising
(a) more than 2.4 mg/ml of a collagen-binding dimeric fusion protein comprising an extracellular domain of glycoprotein VI, the fusion protein having an amino acid sequence of SEQ ID NO: 1, or an amino acid sequence that is at least 99% homologous to the amino acid sequence of SEQ ID NO: 1; and
(b) a pharmaceutically acceptable buffer, the buffer comprising (b1) a buffering component in combination with a pH adjusting agent.

For example, the pharmaceutical composition for use in the prevention or treatment of thrombotic complications according to the present invention is a pharmaceutical composition comprising (a) more than 2.4 mg/ml of a collagen-binding dimeric fusion protein comprising an extracellular domain of glycoprotein VI, the fusion protein having an amino acid sequence of SEQ ID NO: 1, and (b) a pharmaceutically acceptable buffer. Particularly, for example, the (b) buffer comprises (b1) His/HCl, (b2) mannitol, and (b3) sucrose. Preferably, for example, the (b) buffer comprises (b1) His/HCl, (b2) mannitol, (b3) sucrose, and (b4) a detergent selected from Tween 20, Tween 40 or Tween 80.

### Dosage

In a preferred embodiment, the dimeric fusion protein is administered intravenously for the treatment of for thrombotic complications at a dose of from 5 to 300 mg. Preferably, at a dose of from 50 to 250 mg. More preferably, at a dose of from 100 to 200 mg. Even more preferably, at a dose of from 125 to 175 mg. Most preferably, at a dose of about 150 mg. For example, the dimeric fusion protein is administered intravenously for the treatment of for thrombotic complications, such as MI, at a dose of about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg. For example, about 80 mg, about 160 mg, or about 240 mg,

### Single or Multiple Dose

In a preferred embodiment, the pharmaceutical composition for the above-mentioned use is administered as a single dose or multiple doses. A single dose, as used herein, may include single doses as part of a multiple dose regimen within a treatment cycle. In yet another embodiment of the present invention, the composition is administered as a single unit dose. Said multiple doses, as used herein, may be a repeated single dose or single unit dose, wherein each single dose may be followed by a resting period.

In a preferred embodiment, the pharmaceutical composition of the present invention is administered as a single dose. In a particular embodiment, said single dose is a unit dose. Preferably, the dosage of a unit dose is at a dose of from 5 to 300 mg. Preferably, at a dose of from 50 to 250 mg. More preferably, at a dose of from 100 to 200 mg. Even more preferably, at a dose of from 125 to 175 mg. Most preferably, at a dose of about 150 mg. For example, the pharmaceutical composition of the present invention is administered as a single dose at a dose of about 300 mg. For example, the pharmaceutical composition of the present invention is administered as a single dose at a dose of about 80 mg.

In a preferred embodiment, the pharmaceutical composition is administered as a multiple dose regimen. For example, the multiple dose regimen is a time period of approximately 1 day, 2 days, 3 days, 4 days, 7 days, 15 days, 1 month, 2 months, 3 months, or 4 months. For example, the pharmaceutical composition of the present invention is administered as a multiple dose at a dose of about 300 mg in a time period of approximately 0.5 month, 1 month, 2 months, 3 months, or 4 months. For example, the pharmaceutical composition of the present invention is administered in multiple doses at a total dose of about 240 mg with a unit dose of about 80 mg within a time period of approximately 1 day.

### Medical Use For Bleeding Complication

The present invention also relates to a pharmaceutical composition containing a collagen-binding dimeric fusion protein comprising an extracellular domain of glycoprotein VI for use in the prevention or treatment of bleeding complications in a patient by administering the pharmaceutical composition to a patient selected based on a soluble glycoprotein VI concentration in blood plasma of more than 22.8 ng/ml.

In one embodiment, said bleeding complications are associated with the undesired presence of GPVI. Said bleeding complications may be but not limited to bleeding disorders or conditions, such as, for example, bleeding tendency and/or prolonged bleeding time, such as thrombocytopenia such as, for example, idiopathic thrombocytopenia purpura (ITP) or immune thrombocytopenia. In a particular embodiment, said bleeding disorder or condition associated with the undesired presence of GPVI may comprise inflammation and/or cancer.

### Medical Use For Bleeding Complication and Thrombotic Complications

In a preferred embodiment, the patient is also a patient suffering from thrombotic complications. More preferably, the patient is a patient suffering from an acute cardiovascular event selected from the group consisting of acute coronary syndrome (ACS), myocardial infarction (MI), unstable angina pectoris (UAP), acute decompensated heart failure (ADHF), myocardial ischemia, chronic stable angina pectoris, unstable angina pectoris, angioplasty, stroke, transient ischemic attack, claudication(s), vascular occlusion(s), and peripheral artery disease(s). Even more preferably, said acute cardiovascular event is selected from the group consisting of stroke, myocardial infarction (MI) and peripheral artery disease. For example, the patient is a trauma patient, a transplant patient, a cancer patient and/or a patient suffering from cardiovascular disease. For example, the patient is a stroke patient. For example, the pharmaceutical composition as described above is used in the prevention or treatment of bleeding complications such as prolonged bleeding in a patient suffering from acute cardiovascular event such as peripheral artery disease. For example, the pharmaceutical composition as described above is used in the prevention or treatment of prolonged bleedings for a stroke patient, where antiplatelet therapies with a platelet inhibitor such as prasugrel is contraindicated.

### Medical Use For Postoperative Bleeding Complications

In a preferred embodiment, said bleeding complications are postoperative bleeding complications. For example, the pharmaceutical composition as described above is used in the prevention or treatment of bleedings up to 48 hours after surgery or operation. For example, the pharmaceutical composition as described above is used in the prevention or treatment of prolonged bleedings up to 24 hours after surgery or operation.

In a preferred embodiment, said bleeding complications are bleeding complications during anticoagulant therapy. For example, the pharmaceutical composition as described above is used in the prevention or treatment of bleedings in a patient treated by anticoagulant therapy, in particular up to 48 hours after a surgery or operation. For example, the pharmaceutical composition as described above is used in the prevention or treatment of prolonged bleedings up to 24 hours after surgery or operation. The anticoagulant therapy may be a treatment with one or more anticoagulants selected from clopidogrel, warfarin, non vitamin K oral anticoagulants (NOAC) such as rivaroxaban, apixaban, dabigatran or edoxaban and heparin.

In a preferred embodiment, said bleeding complications are bleeding complications after a stroke. For example, the pharmaceutical composition as described above is used in the prevention or treatment of bleedings in a patient having suffered a stroke. For example, the pharmaceutical composition as described above is used in the prevention or treatment of prolonged bleedings up to 24 hours after the stroke

### Medical Use For Bleeding Complication For Percutaneous Intervention or Surgery

In a preferred embodiment, the dimeric fusion protein in the pharmaceutical composition of the present invention is administered prior to a percutaneous intervention or surgery. For example, the pharmaceutical composition as described above is administered by a patient prior to a percutaneous intervention such as percutaneous coronary intervention (PCI) or peripheral artery angioplasty and stenting (PTA). For example, the pharmaceutical composition as described above is administered by a patient prior to surgery, such as coronary artery bypass grafting (CABG) or peripheral artery bypass grafting.

### Medical Use For Bleeding Complication: Patient

In a preferred embodiment, the patient receives additional standard anti-platelet therapy. For example, the pharmaceutical composition as described above is administer by a patient suffering from venous thrombosis and receiving anticoagulation therapy that is problematic with a platelet inhibitor. For example, the pharmaceutical composition as described above is administer by a patient receiving a dual antiplatelet therapy with aspirin and a P2Y12 inhibitor, which is a standard antiplatelet for acute coronary syndromes.

### Medical Use of Fusion Protein of SEQ #1

In a preferred embodiment, the fusion protein of the pharmaceutical composition for use in the prevention or treatment of bleeding complications according to the present invention is a collagen-binding dimeric fusion protein having an amino acid sequence of SEQ ID NO: 1, or an amino acid sequence that is at least 99% homologous to the amino acid sequence of SEQ ID NO: 1. For example, said fusion protein has an amino acid sequence that is at least 90% homologous to the amino acid sequence of SEQ ID NO: 1 and the bleeding complications is bleeding disorders or conditions, such as, bleeding tendency and/or prolonged bleeding time. For example, said fusion protein has an amino acid sequence that is at least 90% homologous to the amino acid sequence of SEQ ID NO: 1 and the patient suffers from idiopathic thrombocytopenia purpura (ITP).

### ELISA

Methods for the determination of the sGPVI concentration in blood plasma form part of common general knowledge and suitable assays may be obtained from commercial sources. In a further preferred embodiment, the sGPVI concentration in blood plasma is determined by a sandwich ELISA method using two GPVI-specific antibodies followed by a peroxidase-conjugated secondary antibody. A suitable assay is provided, e.g. by Invitrogen Cat. Number EH230RB and a method is described, e.g. in Al-Tamini et al., Measuring soluble platelet GPVI in human plasma by ELISA, Platelets, May 2009, 20(3): 143-149. Further, suitable antibodies are disclosed in EP1538165 and EP3793596, or published by Nieswandt B. and Watson S. P, Blood, 102(2), 449-461, (2003) or by Dütting S. et al., Trends in Pharmacological Sciences, 33(11), 583-590, (2012).

The concentration of sGPVI in blood plasma of the patient can be determined by assays. Examples of assays in which a sGPVI concentration in blood plasma, can be determined include, but are not limited to, ELISA, sandwich ELISA, RIA, FRCS, tissue immunohistochemistry, Western-blot, and immunoprecipitation. In a preferred embodiment, the concentration of sGPVI in blood plasma can be detected by an ELISA assay. More preferably, said ELISA assay is a sandwich ELISA assay. Even more preferably, said ELISA assay uses two specific antibodies directed against GPVI and a peroxidase-conjugated secondary antibody.

In a particular embodiment, the patient to be treated by percutaneous intervention or surgery is selected by said diagnostic method as being benefitting from administration of a dimeric fusion protein comprising an extracellular domain of glycoprotein VI for preventing bleeding complications.

### Dosage

In a preferred embodiment, the dimeric fusion protein is administered intravenously for the treatment of for bleeding complications at a dose of from 5 to 300 mg. Preferably, at a dose of from 50 to 250 mg. More preferably, at a dose of from 100 to 200 mg. Even more preferably, at a dose of from 125 to 175 mg. Most preferably, at a dose of about 150 mg. For example, the dimeric fusion protein is administered intravenously for the treatment of for bleeding complications, such as MI, at a dose of about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg. For example, about 80 mg, about 160 mg, or about 240 mg,

### Single or Multiple Dose

In a preferred embodiment, the pharmaceutical composition for the above-mentioned use is administered as a single dose or multiple doses. A single dose, as used herein, may include single doses as part of a multiple dose regimen within a treatment cycle. In yet another embodiment of the present invention, the composition is administered as a single unit dose. Said multiple doses, as used herein, may be a repeated single dose or single unit dose, wherein each single dose may be followed by a resting period.

In a preferred embodiment, the pharmaceutical composition of the present invention is administered as a single dose. In a particular embodiment, said single dose is a unit dose. Preferably, the dosage of a unit dose is at a dose of from 5 to 300 mg. Preferably, at a dose of from 50 to 250 mg. More preferably, at a dose of from 100 to 200 mg. Even more preferably, at a dose of from 125 to 175 mg. Most preferably, at a dose of about 150 mg. For example, the pharmaceutical composition of the present invention is administered as a single dose at a dose of about 300 mg. For example, the pharmaceutical composition of the present invention is administered as a single dose at a dose of about 80 mg.

In a preferred embodiment, the pharmaceutical composition is administered as a multiple dose regimen. For example, the multiple dose regimen is a time period of approximately 1 day, 2 days, 3 days, 4 days, 7 days, 15 days, 1 month, 2 months, 3 months, or 4 months. For example, the pharmaceutical composition of the present invention is administered as a multiple dose at a dose of about 300 mg in a time period of approximately 0.5 month, 1 month, 2 months, 3 months, or 4 months. For example, the pharmaceutical composition of the present invention is administered in multiple doses at a total dose of about 240 mg with a unit dose of about 80 mg within a time period of approximately 1 day.

### Composition For Med Use For Bleeding Complications

In a preferred embodiment, the pharmaceutical composition for use in the prevention or treatment of bleeding complications according to the present invention is a pharmaceutical composition as defined above according to the present invention. Particularly, said pharmaceutical composition for use in the prevention or treatment of bleeding complications according to the present invention is a pharmaceutical composition for parenteral application, comprising
(a) more than 2.4 mg/ml of a collagen-binding dimeric fusion protein comprising an extracellular domain of glycoprotein VI, the fusion protein having an amino acid sequence of SEQ ID NO: 1, or an amino acid sequence that is at least 90%, 91%, 92%, 93%, 94%, 95%, 96%, 97%, 98% or 99% homologous to the amino acid sequence of SEQ ID NO: 1; and
(b) a pharmaceutically acceptable buffer, the buffer comprising (b1) a buffering component in combination with a pH adjusting agent.

For example, the pharmaceutical composition for use in the prevention or treatment of bleeding complications according to the present invention is a pharmaceutical composition comprising (a) more than 2.4 mg/ml of a collagen-binding dimeric fusion protein comprising an extracellular domain of glycoprotein VI, the fusion protein having an amino acid sequence of SEQ ID NO: 1, and (b) a pharmaceutically acceptable buffer. Particularly, for example, the (b) buffer comprises (b1) His/HCl, (b2) mannitol, and (b3) sucrose. Preferably, for example, the (b) buffer comprises (b1) His/HCl, (b2) mannitol, (b3) sucrose, and (b4) a detergent selected from Tween 20, Tween 40 or Tween 80.

### Kit of Parts

The present invention also relates to kit-of-parts comprising a pharmaceutical composition of the present invention, and a diagnostic antibody directed against sGPVI. By “kit” is intended any manufacture (e.g., a package or a container) comprising at least one reagent, i.e. for example an antibody or antibody fragment, for specifically detecting the expression of GPVI. The kit may be promoted, distributed, or sold as a unit for performing the methods of the present invention. Furthermore, any or all of the kit reagents may be provided within containers that protect them from the external environment, such as in sealed containers. The kits may also contain a package insert describing the kit and methods for its use.

The term “diagnostic antibody” refers to an antibody or antibody fragment of the present disclosure labeled for diagnostic or detection purposes. By labeled herein is meant that a compound has at least one element, isotope or chemical compound attached to enable the detection of the compound. For example, the antibody or antibody fragment specific for GPVI can be used for diagnosis of GPVI expression changes. It is described that changes in the expression of GPVI on the platelet surface as well as the occurrence and concentration of soluble GPVI (sGPVI, cleaved extracellular domain of GPVI) in plasma may well be associated with pathophysiological conditions such as acute coronary syndromes, transient ischemic attacks or stroke (Bigalke B, et al., Eur J Neurol., 2009 Jul. 21; Bigalke B. et al., Semin Thromb Hemost. 2007 March; 33(2): 179-84).

Thus, measurement of these parameters could be used to identify subjects at risk for the aforementioned conditions requiring anti-thrombotic treatment and being possibly particularly susceptible for anti-GPVI treatment. Therefore, antibodies and antibody fragments described herein can be used as a diagnostic tool and be part of a diagnostic kit which determines the presence and quantitative changes of GPVI on the platelet surface as well as in plasma samples.

In a particular embodiment, said kit-of-parts is a diagnostic kit which determines the presence and quantitative changes of sGPVI concentration in blood plasma of a patient. For example, said kit-of-parts is a diagnostic kit for selecting a patient benefitting from administration of a dimeric fusion protein comprising an extracellular domain of glycoprotein VI for preventing bleeding complications

### Diagnostic Method

The present invention also relates to a diagnostic method for selecting a patient benefitting from administration of a dimeric fusion protein comprising an extracellular domain of glycoprotein VI for preventing bleeding complications, which method comprises
(a) determining a sGPVI concentration in blood plasma of the patient; and
(b) selecting the patient if the sGPVI concentration is above 22.8 ng/ml. In one embodiment, said bleeding complications are associated with the undesired elevated level of sGPVI. Said bleeding complications may be but not limited to bleeding disorders or conditions, such as, for example, bleeding tendency and/or prolonged bleeding time, such as thrombocytopenia such as, for example, idiopathic thrombocytopenia purpura (ITP) or immune thrombocytopenia. In a particular embodiment, a bleeding disorder or condition associated with the undesired presence of GPVI may comprise inflammation and/or cancer. In a preferred embodiment, said bleeding complications are postoperative bleeding complications. In another preferred embodiment, said bleeding complications are bleeding complications after stroke such as intracranial bleeding complications. In another preferred embodiment, said bleeding complications are bleeding complications in the combination with other anti-coagulant drugs such as rivaroxaban, dabigatran, edoxaban, and apixaban or other novel non vitamin K oral anticoagulants (NOACS) or warfarin and marcumar or other vitamin K antagonists.

### Patient

In a particular embodiment, said patient is to be treated by percutaneous coronary intervention (PCI). In another particular embodiment, said patient is to be treated by coronary artery bypass grafting (CABG). In another particular embodiment, said patient is to be treated by peripheral artery intervention (PTA) or peripheral artery bypass grafting.

In a preferred embodiment, said patient being selected is to be treated by percutaneous intervention or surgery. For example, the patient being selected is to be treated by percutaneous intervention such as percutaneous coronary intervention (PCI) or peripheral artery intervention (PTA). For example, the patient being selected is to be treated by surgery, such as coronary artery bypass grafting (CABG) or peripheral artery bypass grafting.

### ELISA

The concentration of sGPVI in blood plasma of the patient can be determined by assays. Suitable assays form part of common general knowledge or may be purchased from commercial sources, for example Invitrogen Cat. Number EH230RB A general method is described, e.g. in Al-Tamini et al., Measuring soluble platelet GPVI in human plasma by ELISA, Platelets, May 2009, 20(3): 143-149. Examples of assays in which a sGPVI concentration in blood plasma can be determined include, but are not limited to, ELISA, sandwich ELISA, RIA, FRCS, tissue immunohistochemistry, Western-blot, and immunoprecipitation. In a preferred embodiment, the concentration of sGPVI in blood plasma can be detected by an ELISA assay. More preferably, said ELISA assay is a sandwich ELISA assay. Even more preferably, said ELISA assay use two specific antibodies directed against GPVI and a peroxidase-conjugated secondary antibody. antibodies directed against GPVI. Suitable antibodies are disclosed in EP1538165 and EP3793596, or published by Nieswandt B. and Watson S. P, Blood, 102(2), 449-461, (2003) or by Dütting S. et al., Trends in Pharmacological Sciences, 33(11), 583-590, (2012).”

In a particular embodiment, the patient to be treated by percutaneous intervention or surgery is selected by said diagnostic method as being benefitting from administration of a dimeric fusion protein comprising an extracellular domain of glycoprotein VI for preventing bleeding complications.

### Computer Implemented Method

In a preferred embodiment, the diagnostic method of the present invention is computer-implemented. For example, said computer implemented method is provided as a software. For example, said software can be conveniently stored on a non-transitory memory device. In a more preferred embodiment, said computer implemented method is provided for determining the sGPVI concentration in blood plasma of the patient. For example, the instrument for measuring the sGPVI concentration in blood plasma is controlled by a software. For example, the calculation of a sGPVI concentration in blood plasma is provided by a software. In a more preferred embodiment, said computer implemented method is provided for classifying a patient as either or not benefitting from administration of a dimeric fusion protein comprising an extracellular domain of glycoprotein VI for preventing bleeding complications. In an even more preferred embodiment, said computer implemented method is provided for selecting the patient as being benefitting from administration of a dimeric fusion protein comprising an extracellular domain of glycoprotein VI for preventing bleeding complications. For example, said software selects the patient, if the sGPVI concentration is above 22.8 ng/ml.

### WORKING EXAMPLES

### Example 1 Collagen Binding Dimeric Fusion Protein

One working example of the collagen-binding dimeric fusion protein as described in the present disclosure is Human Fc fusion protein Revacept® (PR-15, GPVI-Fc). GPVI-Fc is known to be a fusion protein consisting of the extracellular domain (receptor) of glycoprotein VI fused to an Fc region. GPVI-Fc has an amino acid sequence of SEQ ID NO: 1. The isoelectric point of GPVI-Fc is experimentally determined to be 4.2-5.2. The molecular mass of monomeric GPVI-Fc is ˜80 kDa under reducing conditions in SDS-PAGE, as detected by Coomassie blue stain or by immunoblotting with peroxidase-conjugated goat anti-human Fc antibody or by the anti-GPVI mAb 5C4. In contrast, the molecular weight of GPVI-Fc of the present invention as identified under nonreducing conditions is 150 kDa, which indicates that GPVI-Fc is present as dimer. GPVI-Fc is known to bind specifically to collagen.

### Example 2 Pharmaceutical Composition Comprising GPVI-Fc

GPVI-Fc can be formulated as a liquid formulation for parenteral application. A list of exemplary formulations is summarized in the Table 4. These formulations are pharmaceutically acceptable for intravenous administration, e.g. via infusion with a perfusor syringe.

Following are some more specific examples.

### Example 2.1 Formulation #24 of GPVI-Fc

Route of administration: i.v. for infusion (perfusor syringe)

2.4 mg/mL Revacept DS (GPVI-Fc) in DPBS, 4% mannitol, 1% sucrose, pH 7.4. Said formulation is stable for long-term storage when frozen at −80° C. or −25° C. A further variation based on formulation #24 comprise Tween 20 as a detergent. Tween 20 does not affect long-term stability of the formulation.

### Example 2.2 Formulation #26 of GPVI-Fc

5 mg/mL Revacept DS (GPVI-Fc) in 10 mM Histidine/HCl buffer, 4% mannitol, 2.5% sucrose, pH 7.0.

This formulation is packaged in 20 mL glass vials, with a dosage of 80 mg per vial, (fill volume 16.6 mL) A maximum dosage of 240 mg Revacept DS (GPVI-Fc) can be achieved by combining the contents of 3 vials into a 50 ml perfusor syringe. Formulation #26 is stable for long-term storage when frozen at −20° C. for up to 2 years. Formulation #26 is stable for long-term storage at 2-8° C. for up to 2 years. Formulation #26 can be freeze dried and the lyophilized formulation #26 is stable for long-term storage at 25° C. for up to 2 years. The lyophilized formulation #26 can be rehydrated and being stable for long-term storage at 2-8° C. for up to 2 years. A further variation based on formulation #26 comprise Tween 20 as a detergent. Tween 20 does not affect long-term stability of the formulation.

### Example 2.3. Formulation #31 of GPVI-Fc

5 mg/mL Revacept DS (GPVI-Fc) in 10 mM Tris/Acetic acid buffer, 4% mannitol, 2.5% sucrose, pH 8.0.

This formulation is packaged in 20 mL glass vials, with a dosage of 80 mg per vial, (fill volume 16.6 mL) A maximum dosage of 240 mg Revacept DS (GPVI-Fc) can be achieved by combining the contents of 3 vials into a 50 ml perfusor syringe.

Formulation #31 is stable for long-term storage when frozen at −20° C. for up to 2 years. Formulation #31 is stable for long-term storage at 2-8° C. for up to 2 years. Formulation #31 can be freeze dried and the lyophilized formulation #31 is stable for long-term storage at 25° C. for up to 2 years. The lyophilized formulation #31 can be rehydrated and being stable for long-term storage at 2-8° C. for up to 2 years. A further variation based on formulation #31 comprise Tween 20 as a detergent. Tween 20 does not affect long-term stability of the formulation.

### Example 3. Comparison of Variants in Formulations

Various formulation systems were screened via HTS for determination of thermodynamic stability (determination of Tₒₙₛₑₜ) and colloidal stability (determination of attractive/repulsive interactions of the protein, reflected as k_{D} value) via DLS. The tables of variants are shown in Table 4. The k_{D} measurements as a clear negative value indicates strong attractive interactions. Neutral interactions are shown by values around 0, clearly repulsive interactions are shown by positive values clearly above zero. All DLS results are concluded in Table 4.

In a first step, a DLS measurements series with different generic buffer systems, with and without NaCl, was measured (Series 1, variants #1-#11). This series resulted in only two variants showing positive k_{D} values, representing repulsive protein-protein-interaction: variant #4 (His/HCl, pH 7.0) and variant #10 (Tris/HCl, pH 8.0). The corresponding variants with NaCl (#5 and #11) resulted in lower k_{D} values, indicating that higher ionic strength was not favorable in these buffer systems. Low performing buffer systems often showed opposite results and high ionic strength variants (with NaCl) showed better results as their corresponding variants without NaCl (e.g., variants #2/#3 and #6/#7).

In summary, the data indicates that a high ionic strength seems to be suboptimal for the protein in the better performing formulation variants (#4 and #10). In addition, all variants with phosphoric acid showed negative k_{D} values, indicating attractive protein-protein interactions. The Tₒₙₛₑₜ values were overall acceptable, as Revacept denaturation by unfolding is not an issue below a thermal stress of 54° C. However, variant #4 (His/HCl, pH 7.0) and variant #10 (Tris/HCl, pH 8.0) showed the highest Tₒₙₛₑₜ values.

In a second step (Series #2, variants #12-#23), first of all it was tested if acetic acid is more suitable than HCl for adjusting pH in the best variants from Series 1 (#4 and #10). With the first two variants of measurement Series 2 (#12-#13) His/acetic acid and Tris/acetic acid were analyzed to compare them with the corresponding variants prepared with HCl. The two favorites of the four variants were His/HCl PH 7.0 and Tris/acetic acid pH 8.0 considering k_{D} and Tₒₙₛₑₜ. Based on these results, potential stabilizers were screened for His/HCl PH 7.0 (#14-#18) and Tris/acetic acid pH 8.0 (#19-#23).

Regarding the variants of measurement with potential stabilizers, variants #16 (His/HCl, pH 7.0, 5% mannitol) and #18 (His/HCl, pH 7.0, 150 mM proline) resulted best considering k_{D} and Tₒₙₛₑₜ. The ionic attributes of arginine and the corresponding negative influence on the k_{D} was confirmed (variants #17 and #22), as already seen in Series 1 in the variants containing NaCl (#5 and #11). As already assumed, due to the problems occurring during the concentration process, also the current formulation (variant #24 with PBS, 4% mannitol, 1% sucrose) performed suboptimal in respect to colloidal stability (negative k_{D}). Overall, variants with Tris/acetic acid showed lower colloidal stability but higher Tₒₙₛₑₜ compared to variants with His/HCl acid.

TABLE 3
Potential stabilizer abilities regarding
processability and storage conditions

| | | Potential stabilizer |
|---|---|---|
| | | abilities regarding |
| | | processability and |
| Variant | Potential stabilizer | storage conditions |
| #14 | 5% trehalose | Liquid (2-8° C.; |
| | | −20° C.) + Lyoph. |
| #15 | 5% sucrose | Liquid (2-8° C.; |
| | | −20° C.) + Lyoph. |
| #16 | 5% mannitol | Liquid (2-8° C.; |
| | | −20° C.) + Lyoph. |
| #17 | 150 mM arginine | Liquid (2-8° C.; |
| | | −20° C.) + Lyoph. |
| #18 | 150 mM proline | Liquid (2-8° C.; |
| | | −20° C.) |
| #19 | 5% trehalose | Liquid (2-8° C.; |
| | | −20° C.) + Lyoph. |
| #20 | 5% sucrose | Liquid (2-8° C.; |
| | | −20° C.) + Lyoph. |
| #21 | 5% mannitol | Liquid (2-8° C.; |
| | | −20° C.) + Lyoph. |
| #22 | 150 mM arginine | Liquid (2-8° C.; |
| | | −20° C.) + Lyoph. |
| #23 | 150 mM proline | Liquid (2-8° C.; |
| | | −20° C.) |
| #24 | 4% mannitol 1% sucrose | Liquid (2-8° C.; |
| | | −20° C.) + Lyoph. |
| #25 | 2.5% mannitol 5% sucrose | Liquid (2-8° C.; |
| | | −20° C.) |
| #26 | 4% mannitol 2.5% sucrose | Liquid (2-8° C.; |
| | | −20° C.) + Lyoph. |
| #27 | 300 mM proline | Liquid (2-8° C.; |
| | | −20° C.) |
| #28 | 2.5% mannitol 150 mM proline | Liquid (2-8° C.; |
| | | −20° C.) + Lyoph. |
| #29 | 2.5% mannitol 5% sucrose | Liquid (2-8° C.; |
| | | −20° C.) |
| #30 | 4% mannitol 2.5% sucrose | Liquid (2-8° C.; |
| | | −20° C.) + Lyoph. |
| #31 | 4% mannitol 2.5% sucrose | Liquid (2-8° C.; |
| | | −20° C.) + Lyoph. |
| #32 | 2.5% mannitol 150 mM proline | Liquid (2-8° C.; |
| | | −20° C.) + Lyoph. |

TABLE 4
Results of Revacept high−throughput screening
with DLS (buffer and stabilizer screening) k_{D} nm

| | | | pH | | k_{D} | | |
|---|---|---|---|---|---|---|---|
| | | | adjustment | | mL/mg | Tₒₙₛₑₜ | Rₕ₀ |
| Variant | Buffering comp. | Stabilizer | component | ph | (10⁻³) | ° C. | nm |
| #1 | 50 mM phosphoric | 150 NaCl | NaOH | 7.4 | −5.2 | 55.2 | 6.6 |
| | acid | | | | | | |
| #2 | 10 mM phosphoric | | KOH | 7.0 | −20.1 | 56.3 | 5.9 |
| | acid | | | | | | |
| #3 | 10 mM phosphoric | 150 NaCl | KOH | 7.0 | −6.5 | 54.6 | 6.4 |
| | acid | | | | | | |
| #4 | 10 mM Histidine | | HCl | 7.0 | 126.0 | 63.4 | 4.7 |
| #5 | 10 mM Histidine | 150 NaCl | HCl | 7.0 | −5.5 | 54.0 | 6.5 |
| #6 | 10 mM phosphoric | | KOH | 7.5 | −25.8 | 56.7 | 6.1 |
| | acid | | | | | | |
| #7 | 10 mM phosphoric | 150 NaCl | KOH | 7.5 | −5.8 | 57.3 | 6.5 |
| | acid | | | | | | |
| #8 | 10 mM Tris | | HCl | 7.5 | −2.3 | 57.0 | 5.8 |
| #9 | 10 mM Tris | 150 NaCl | HCl | 7.5 | −4.2 | 57.7 | 6.5 |
| #10 | 10 mM Tris | | HCl | 8.0 | 31.9 | 58.1 | 5.8 |
| #11 | 10 mM Tris | 150 NaCl | HCl | 8.0 | −8.8 | 56.5 | 6.4 |
| #12 | 10 mM Histidine | | acetic acid | 7.0 | 127.0 | 55.7 | 4.2 |
| #13 | 10 mM Tris | | acetic acid | 8.0 | 40.8 | 60.8 | 5.8 |
| #14 | 10 mM Histidine | 5% | HCl | 7.0 | 65.8 | 53.5 | 3.4 |
| | | trehalose | | | | | |
| #15 | 10 mM Histidine | 5% sucrose | HCl | 7.0 | 104.0 | 54.6 | 3.5 |
| #16 | 10 mM Histidine | 5% mannitol | HCl | 7.0 | 145.0 | 54.8 | 4.3 |
| #17 | 10 mM Histidine | 150 mM | HCl | 7.0 | −22.6 | 53.6 | 6.3 |
| | | arginine | | | | | |
| #18 | 10 mM Histidine | 150 mM | HCl | 7.0 | 155.0 | 54.2 | 4.6 |
| | | proline | | | | | |
| #19 | 10 mM Tris | 5% | acetic acid | 8.0 | −19.9 | 61.7 | 5.4 |
| | | trehalose | | | | | |
| #20 | 10 mM Tris | 5% sucrose | acetic acid | 8.0 | 10.4 | 59.8 | 5.8 |
| #21 | 10 mM Tris | 5% mannitol | acetic acid | 8.0 | 17.1 | 60.0 | 6.3 |
| #22 | 10 mM Tris | 150 mM | acetic acid | 8.0 | −16.3 | 55.6 | 6.7 |
| | | arginine | | | | | |
| #23 | 10 mM Tris | 150 mM | acetic acid | 8.0 | 27.5 | 59.4 | 5.9 |
| | | proline | | | | | |
| #24 | DPBS | 4% mannitol | DPBS | 7.4 | −17.9 | 55.9 | 6.9 |
| | | 1% sucrose | | | | | |
| #25 | 10 mM Histidine | 2.5% | HCl | 7.0 | 60.3 | 55.7 | 3.6 |
| | | mannitol 5% | | | | | |
| | | sucrose | | | | | |
| #26 | 10 mM Histidine | 4% mannitol | HCl | 7.0 | 92.4 | 55.9 | 4.1 |
| | | 2.5% | | | | | |
| | | sucrose | | | | | |
| #27 | 10 mM Histidine | 300 mM | HCl | 7.0 | 141.0 | 56.4 | 4.7 |
| | | proline | | | | | |
| #28 | 10 mM Histidine | 2.5% | HCl | 7.0 | 120.0 | 53.6 | 4.2 |
| | | mannitol 150 | uses | | | | |
| | | mM proline | | | | | |
| | | we | | | | | |
| #29 | 10 mM Tris | 2.5% | acetic acid | 7.5 | −13.2 | 59.0 | 6.3 |
| | | mannitol 5% | | | | | |
| | | sucrose | | | | | |
| #30 | 10 mM Tris | 4% mannitol | acetic acid | 7.5 | −4.0 | 58.2 | 6.5 |
| | | 2.5% | | | | | |
| | | sucrose | | | | | |
| #31 | 10 mM Tris | 4% mannitol | acetic acid | 8.0 | 16.9 | 60.6 | 6.1 |
| | | 2.5% | | | | | |
| | | sucrose | | | | | |
| #32 | 10 mM Tris | 2.5% | acetic acid | 7.5 | −8.8 | 57.8 | 6.2 |
| | | mannitol 150 | | | | | |
| | | mM proline | | | | | |

Based on the results obtained in this WP2 and considering the prerequisite to find a frozen liquid stable formulation, the most promising formulation is variants #26 (10 mM His/HCl, 4% mannitol, 2.5% sucrose, pH 7.0). This variant is as close as possible to the current formulation, allows to compare influence of pH, is suitable for frozen liquid formulation (sucrose acting a cryoprotectant) and suitable for lyophilization (due to the combination of sucrose and mannitol).

### Example 4. Methods for High-Throughput Screening (HTS)

High-throughput screening (HTS) is applied to evaluate the colloidal and thermodynamic stability of formulation. Variants include buffer types, ionic strength and stabilizer/excipient levels, and suitable conditions. All variants are prepared (by dialysis) and analyzed. Variants are tested with respect to their relevance for stabilizing the API in the pharmaceutical composition.

HTS is performed using dynamic laser light scattering (DLS) measurements in a DLS-plate reader with respect to increasing the colloidal and thermodynamic stability of the formulation. k_{D} and Tₒₙₛₑₜ are measured. The DS was to be dialyzed against different formulation buffer systems. If needed, the DS was to be concentrated to 10-15 mg/mL prior dialysis, to gain suitably concentrated samples for DLS measurements. Protein-protein interactions as a measure for the colloidal stability in each formulation buffer system were to be determined by measuring the hydrodynamic radius of the DS with increasing concentration of the DS. The data was to be used to predict formulation candidates holding a lower risk of protein aggregation during handling and storage. Denaturation temperature (thermodynamic stability) in each formulation buffer system was to be determined by measuring the hydrodynamic radius of the DS with increasing temperature. Once protein domains begin to denature, the hydrodynamic radius of the protein rises remarkably. The onset-temperature of unfolding can be taken as an indicator of the secondary structure stability of the protein. The data was to be used to predict formulation candidates holding a lower risk of protein denaturation during handling and storage.

Substances and materials used in the HTS are listed in Table 1. The apparatus and equipment used are listed in Table 2.

TABLE 1
Substances and materials for HTS

| Substances | Lot-no. | Supplier | Order no. |
|---|---|---|---|
| Revacept DS; 2.4 mg/mL; | CAD/02D | AdvanceCor | — |
| 40 mg/vial; in PBS, | | | |
| 1% sucrose, | | | |
| 4% mannitol, pH 7.4 | | | |
| acetic acid, 100%, | 359283308 | Carl Roth | 6755.1 |
| Ph. Eur. | | GmbH | |
| D(−)-mannitol, | 509290856 | Carl Roth | 4175 |
| ≥98%, Ph. Eur., USP | | GmbH | |
| hydrochloric acid 25%, | Z0395312 | Merck | 100312 |
| EMPROVE ® exp Ph. | | Chemicals | |
| Helv. | | | |
| L-Arginine | 060283900 | Carl Roth | 3145.2 |
| monohydrochloride | | GmbH | |
| L-Histidine, Ph. Eur. | 146239022 | Carl Roth | 3852.2 |
| | | GmbH | |
| L-Proline, ≥98.5%, | 388272032 | Carl Roth | T205 |
| Ph. Eur. | | GmbH | |
| ortho phosphoric acid, | 306246347 | Carl Roth | 2608.1 |
| 85%, Ph. Eur. | | GmbH | |
| potassium hydroxide, | 076230106 | Carl Roth | P747.2 |
| ≥85%, Ph. Eur. | | GmbH | |
| sodium chloride, | K45515600 | Merck | 106400 |
| EMPROVE ® exp Ph. | | Chemicals | |
| Eur., BP, USP | | | |
| Sodium hydroxide | 0000955695 | AppliChem | 144320.1211 |
| solution, 30% w/w | | | |
| pure, pharma grade | | | |
| sucrose, EMPROVE ® | K42784453205 | Merck | 107653 |
| exp, Ph. Eur. | | Chemicals | |
| trehalose dihydrate | 8K191 | Hayashibara | 33016 |
| 100 PH (pharma grade) | | | |
| TRIS, ≥99%, Ph. | 378264352 | Carl Roth | A411.1 |
| Eur., USP | | GmbH | |
| demineralized water, | / | PJP | / |
| <0.2 μS/cm | | | |
| Molecular porous | 3248816 | Spectrum | 132650 |
| membrane tubing | 9200685 | Laboratories | 132665 |
| 6-8 kDa MWCO | | | |
| Spectra/Por ® | | | |
| dialysis membrane | | | |
| Amicon ® Ultra -15 | R9CA80361 | Merck | UFC903024 |
| Centrifugal Filters | | Millipore | |
| Regenerated Cellulose | | | |
| 30 kDa MWCO | | | |
| Minisart Syringe | 91076103 | Sartorius | 16553 |
| Filter, 0.1 μm pore | | | |
| size, PES | | | |
| Microplate 384-round- | 506301708017 | Aurora | — |
| well LV, black, | | | |
| Silicone oil M1000 | 180156774 | Carl Roth | 4045.1 |

TABLE 2
Apparatus and equipment

| Equipment | Manufacturer: | Type: |
|---|---|---|
| DLS plate reader | Wyatt Technology Corporation | Dynapro |
| | (Santa Barbara, CA, USA) | |
| plate reader II Liquid | Eppendorf AG | epmotion 5070 |
| handling station | (Hamburg, Germany) | |
| UV Spectrophotometer | Thermo Fisher Scientific | NanoDrop One |
| | (Waltham, MA, USA) | |
| Purified water supply | Evoqua | Ultra Clean UV |
| | (Günzburg, Germany) | UF TM |

### Concentration by UV 280 Measurement

The content of Revacept in aqueous solutions was determined by UV 280 nm absorption measurement using a Thermo NanoDrop spectrophotometer. The absorption (A) of the sample was measured at λ=280 nm with a variable path length between 1 mm and 0.2 mm. Samples were analyzed undiluted. The corresponding buffer served as blank. The concentration (c) of the formulation was calculated using Beer-Lambert's law

### Dialysis

Samples were prepared by dialyzing the bulk drug substance (BDS) into the selected buffer variants: After the first trials of concentrating Revacept in the current formulation buffer (PBS, 1% sucrose, 4% mannitol, pH 7.4) up to 5-15 mg/mL using centrifugal filters, substantial losses of about 50% were observed. The first DLS measurements showed good k_{D} results using a formulation with histidine/HCl pH 7.0. Therefore, for the following concentration steps, the original formulation was first dialyzed against the 100-fold sample volume of histidine/HCl PH 7.0 formulation buffer. The losses were then in a usual and acceptable range for this buffer variant. Revacept was concentrated to 5-15 mg/mL and about 1.5-2.0 mL of the concentrated solution were transferred into pre-conditioned (with target buffer) dialysis tubes and dialyzed in three steps, each against 200 mL of the target buffer. Every dialysis step was performed under gentle stirring for at least 3 h at 2-8° C. The dialyzed sample was removed from the dialysis tubes. Finally, buffer and dialyzed samples were filtrated through a 0.1 μm filter.

### Determination of the Protein-Protein Interaction by DLS

At higher concentration of macromolecules, interactions between adjacent particles result in non-ideal diffusion behavior (intermolecular stability gets affected). This can be described by the second hydrodynamic virial coefficient k_{D}. The k_{D}-value describes the propensity for nonspecific molecular association under a given set of solution conditions. The k_{D}-value can be determined by a first order polynomic fit of the changed diffusion coefficient vs. changing concentration.

Higher diffusion coefficient correlates with higher concentration:

Read-out: Positive increase of line accounting for repulsive molecule-molecule interactions (tendency for aggregation low; higher colloidal stability). The higher (more positive) the increase, the higher (more positive) the k_{D} value. A higher diffusion coefficient k_{D} indicates that dissolved molecules in the solution appear with a smaller Rₕ (hydrodynamic radius), and thus are subject to a faster diffusion.

Lower diffusion coefficient with higher concentration:

Read-out: Decrease of line accounting for attractive molecule-molecule interactions (tendency for aggregation in principle higher; lower colloidal stability). A smaller diffusion coefficient k_{D} indicates that dissolved molecules in the solution appear with a higher Rₕ (hydrodynamic radius), and thus are subject to a slower diffusion.

### Determination of the Denaturation Temperature by DLS

The Tₒₙₛₑₜ (denaturation onset temperature-the starting point of the unfolding transition) is determined by measuring the hydrodynamic radius over temperature. Native proteins respond to heating by unfolding (thermal denaturation) at a characteristic temperature (Tₘₐₓ). The more intrinsically stable the biopolymer is the higher is the starting temperature of the unfolding transition. By increasing temperature of the sample in solution, the start of unfolding of protein domain(s) can be monitored by an increase in the molecule's hydrodynamic radius. Via unfolding the intramolecular stability gets affected, various charges etc. get exposed to the molecule's outside, which amongst other effects promote aggregation.

Read-out: the sooner and steeper the increase of the hydrodynamic radius over temperature in the respective solvent, the lower the thermodynamic stability of a molecule.

### DLS Sample Measurement

After dialysis, all samples and buffers were passed through a 0.1 μm syringe filter. The exact concentration of the sample was determined by UV-absorption measurement. For each sample, four sets of ten equidistant dilution steps were prepared. Protein-protein interaction: The samples were equilibrated to 25° C. for 1 minute. The DLS acquisition time was set to 5 seconds for every 5 acquisitions per well.

Denaturation temperature: The temperature gradient was set after the measurement of all dilutions for protein-protein interaction. The temperature gradient was set from 25° C. to 85° C. with a temperature rate of 0.21° C./min. The DLS acquisition time was set to 5 seconds. The number of DLS acquisitions per well was 5 measurements per well per ° C. The denaturation temperature was determined at the highest available sample concentration.

### Example 5. Medical Use of GPVI-Fc Composition in Treating Thrombotic Complications and/or Bleeding Complications

The Revacept/CS/02 study (NCT01645306) was an international, prospective, randomized, placebo-controlled, double-blind explorative phase II study that prospectively included patients with recent ischemic stroke/TIA due to symptomatic ICA stenosis. Patients were enrolled in 16 centers in the United Kingdom and Germany from 8 Mar. 2013 until 27 Sep. 2018.

In summary, Inhibition of platelet activation via GPVI in patients with symptomatic ICA stenosis safely reduces cerebrovascular events, opening the therapeutic avenue for administration of Revacept as an additional platelet inhibitory medication in other arterial thromboembolic diseases such as myocardial infarction and peripheral artery disease.

### Patients

Patients were included, if older than 18 years with symptomatic, extracranial ICA stenosis presenting with ischemic stroke, TIA or intermittent blindness (amaurosis fugax) within the last 30 days and at least 50% stenosis of the ICA according to European Carotid Surgery Trial (ECST) duplex criteria. Exclusion criteria included those taking dual antiplatelets, oral anticoagulation or who had received intravenous thrombolysis within the last 48 hours before screening. Other exclusions were those with concurrent cardiac cause of stroke (e.g. atrial fibrillation), recent intracranial hemorrhage, uncontrolled hypertension (systolic blood pressure above 179 mmHg or diastolic blood pressure above 109 mmHg), National Institute of Health Stroke Scale (NIH-SS) above 18 and no acoustical window available for transcranial Doppler sonography.

### Randomization and Masking

Eligible subjects were randomized 1:1:1 by the local investigator to one of three treatment groups: placebo, Revacept 40 or Revacept 120 mg by using a minimized randomization method in order to balance potential prognostic factors between individual treatment. This was achieved using the web-based, independent, secure and validated randomization tool randomizer.at provided by Medical University of Graz, Institute for Medical Informatics, Statistics and Documentation (IMI). The following stratification factors were used during treatment allocation: 1.) Patient has received anti-platelet therapy with aspirin or Clopidogrel prior to screening (Yes/No), 2.) Patient has received statin therapy prior to screening (Yes/No), 3.) Degree of carotid stenosis (50-70%/<70%). Treating physicians, patients and study personal assessing outcomes (evaluation of MES and number of DWI-lesions) were blinded to treatment groups.

### Procedures

Study medication, manufactured and provided by advanceCOR (Martinsried, Germany), was administered by a single intravenous infusion over 20 minutes in 50 ml volume via an infusion pump. Follow-ups were scheduled one and three days after study drug administration, and after 3 and 12 months. At screening, patients underwent a structural interview concerning medical history, physical examination, laboratory assessment, electrocardiogram (ECG), transcranial Doppler sonography and MR-imaging with a standardized DWI-acquisition. At each follow-up visit, patients were assessed for concomitant medication and occurrence of adverse events. Clinical outcomes (any stroke, TIA, coronary events or bleeding complications) were assessed via a clinical visit one and three days after study drug administration and after 3 months; a telephone interview was conducted after 12 months. To assess an effect of Revacept on MES and DWI-MRI the procedures were scheduled as follows: 1) transcranial Doppler examination for MES-evaluation was performed at screening and repeated one day after study drug administration; 2) MRI for evaluation of new DWI lesions was performed at screening and repeated 24 hours after the revascularization procedure (CAS, CEA).

### MES-Detection by TCD

All TCD-recordings were obtained from the middle cerebral artery (MCA) with a DWL TCD machine (Compumedics GmbH, Singen, Germany) with a single-depth 2-MHz transducer. Standard settings were used by all study centers. (Ringelstein EB et al, International Consensus Group on Microembolus Detection. Stroke; a journal of cerebral circulation. 1998; 29(3):725-9) Patients were placed in a sitting or supine position for TCD recordings. MCA was identified through a transtemporal window and transducer was attached using a standard headset. TCD signals were recorded for one hour prior to application of study drug (screening visit) and for up to 24 hours after study drug administration. After completion of a sufficient test run, the total number of MES per hour was analyzed by a central core laboratory, led by M. Ritter (Münster, Germany), blinded to clinical data and treatment group. At start of the study period, the detection of MES by transcranial Doppler was an inclusion criterion. Due to the low MES-incidence in patients screened for potential study participation resulting in low recruitment rates, this criterion was not mandatory for randomization with protocol version 8 (dated 22 Jun. 2015).

### DWI

MRI studies were performed on a 1.5 or 3 Tesla imaging system. Whole brain DWI was carried out using coronal and transversal studies, each with b values of 0 and 1000 s/mm², TR (repetition time) 4006 ms, TE (echo time) 83 ms, quantum gradient 30 mT/m, slew rate 125 mT/m, rising time 240 ms and apparent diffusion coefficient (ADC) maps. Slice thickness ≤6 mm, gap ≤1.5 mm, 128×128 matrix and 22×220 mm² field of view. The diffusion-weighted data were automatically processed by the scanner's software in order to minimize the effects of diffusion anisotropy. ADC maps were also automatically processed by the scanner's software. Image acquisition at baseline and follow-up was acquired by the same scanner. All images were analyzed by a core laboratory (T-K. Hauser, Tübingen, Germany) blinded to clinical details and treatment group. An acute ischemic lesion in DWI was diagnosed only if increased signal intensity was visible on at least two planes, and a corresponding decreased signal intensity was detected on the ADC image.

### Outcomes

The study was conducted as an exploratory study. The efficacy endpoint was new lesions on DWI-MRI. DWI-MRI lesions were assessed in a central reading lab by an experienced neuroradiologist (TKH) in a blinded fashion directly comparing the initial DWI-MRI images with the follow-up imaging in a head-to-head manner. As predefined, clinical endpoints are considered cumulatively including a combined efficacy and safety endpoint of any cerebrovascular events (ischemic stroke, hemorrhagic stroke, TIA, myocardial infarction or coronary intervention) or bleeding complications. Safety endpoints (bleeding complications) were defined according to the RE-LY-Study (Connolly S J et al. The New England journal of medicine. 2009; 361(12):1139-51.) (fall in hemoglobin of at least 20 g/liter, transfusion of at least two units of blood, symptomatic bleeding in a critical area or organ) and also incorporated bleedings rated as adverse event by the local investigator. As mentioned above, due to the low MES-incidence MES no longer served as inclusion criterion and the reduction of MES no longer served as primary endpoint. The study was changed to an exploratory study of the previously defined endpoints with the number of new DWI-MRI lesions as one of the main efficacy endpoints.

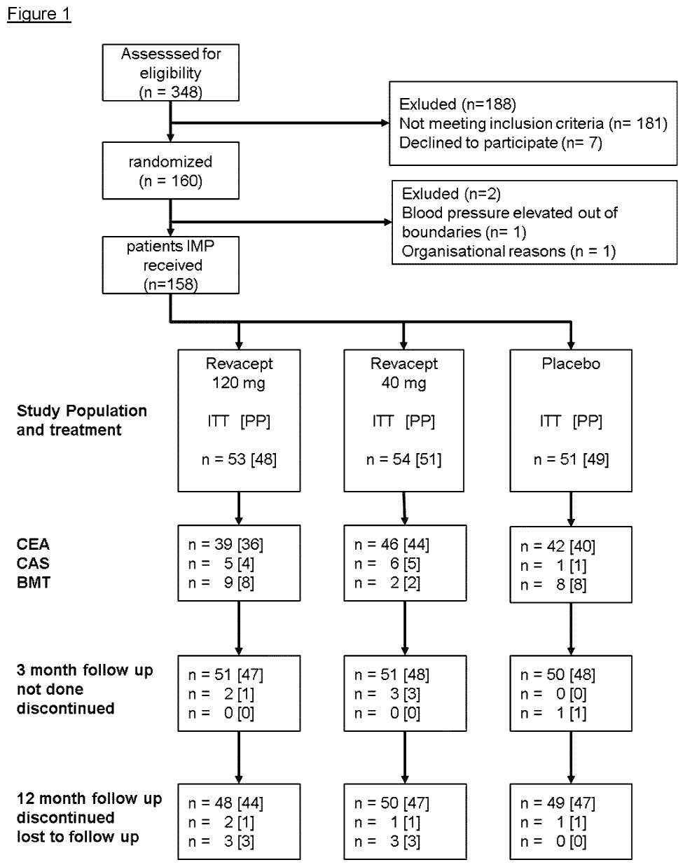

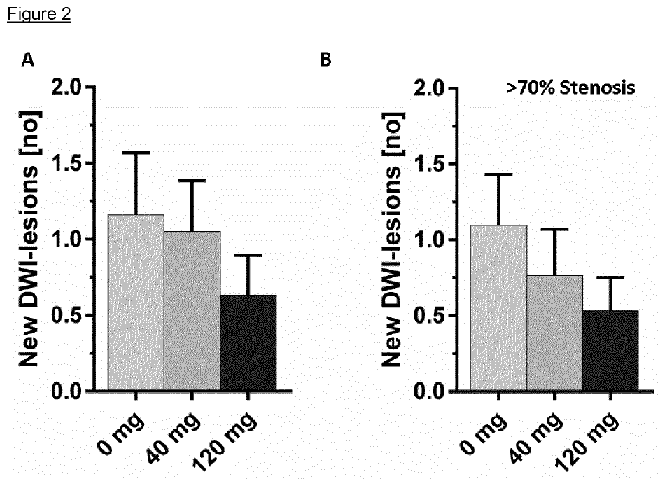

### Results of FIG. 1-5

From 8 Mar. 2013 until 27 Sep. 2018, 348 patients were screened for eligibility. 188 patients were not included in the study due to not fulfilling inclusion criteria (n=181) or declining to participate (n=7). Thus, 160 patients were included in the Revacept/CS/02 study and were randomized to one of the three treatment groups (placebo: n=51, 40 mg Revacept n=56, 120 mg Revacept n=53). After randomization, two additional patients were excluded due to elevated blood pressure (n=1) and organizational reasons (n=1), both impeding study drug administration, so that ultimately 158 patients received study medication and served as patient population for intention-to-treat analysis (ITT, placebo n=51, 40 mg Revacept n=54, 120 mg Revacept n=53). The frequency of patients within the three treatment groups within 16 recruiting centers is given in Supplemental Table 1. MRI at Baseline was available in 139 patients (placebo n=46, Revacept 40 mg n=46, Revacept 120 mg n=47), whereas MRI at two time points was available in 126 patients (placebo n=44, Revacept 40 mg n=41, Revacept 120 mg n=41). The patient population with MRI-scans available at two time points were assessed with regard to development of new DWI-lesions. All 158 patients were assessed with regard to cerebrovascular events (ischemic stroke, hemorrhagic stroke, TIA, myocardial infarction, coronary intervention) or bleeding complications. In case of study discontinuation (placebo n=1, Revacept 40 mg n=1, Revacept 120 mg n=2) or loss to follow-up (placebo n=0, Revacept 40 mg n=3, Revacept 120 mg n=3) the patients were censored at last available follow-up for assessment of the clinical endpoints mentioned above. Details are depicted within the consort diagram (FIG. 1).

The study population had a mean age of 68 years (SD 10.1, range 61-76). 38 of 158 patients (24%) were women; mean duration of follow-up was 11.2 +2.3 months. Detailed patient characteristics at screening are shown in Table 1. Patients had common risk factors for arteriosclerosis such as arterial hypertension (n=105, 66.5%), diabetes mellitus (n=37, 23.4%), hyperlipidemia (n=62, 39.3%) and current smoking (n=49, 31.0%). These cardiovascular risk factors and their associated cardiovascular diseases such as previous ischemic stroke (n=25, 15.8%), TIA (n=18, 11.4%), coronary artery disease (n=29, 18.4%) and peripheral artery disease (n=16, 10.1%) were reflected by a frequent use of antiplatelet therapy (n=137, 86.7%) and statins (n=130, 82.3%). Stroke severity as measured by National Institute of Health Stroke Scale (NIH-SS) and modified Rankin Scale (mRS) was equally distributed between the three treatment groups. In addition, number of MES/h and DWI lesions at baseline were evenly distributed between the three treatment groups, for details see Table 2.

Degree of symptomatic ICA stenosis was rated by ultrasound according to ECST-criteria, with a distribution of 42 patients (26.6%) with 50-70% stenosis and 116 patients (73.4%) with a stenosis above 70% (equivalent to ICA stenosis above 50% according to NASCET-criteria) (Arning C et al, Ultraschall Med. 2010; 31(3):251-7). With regard to management of the symptomatic ICA stenosis, 127 patients (80.4%) underwent CEA, 12 patients (7.6%) were revascularized by CAS and 19 patients (12.0%) received BMT. The management of the ICA stenosis was at the discretion of the attending physician. As demonstrated, above randomization formed balanced treatment groups. However, CAS tended to be more frequent in 40 mg (6/54, 11.1%) and 120 mg Revacept (5/53, 9.4%) compared to Placebo (1/51, 2.0, p=0.173), whereas BMT was more frequently found in Placebo (8/51, 15.7%) and 120 mg Revacept (9/53, 17.0%) compared to 40 mg Placebo (2/54, 3.7%, p=0.067, for details see Table 1).

Evaluation of MES was limited as around 50% (n=79) of all study patients presented with any MES at screening. With regard to treatment groups, MES prevalence at screening was as follows: Placebo 26/48 (54.2%), Revacept 40 mg 26/46 (56.5%), Revacept 120 mg 27/45 (60%). The mean number of MES signals was similar between different treatment groups both before (placebo 5.9+12.8, 40 mg Revacept 5.2±10.9, 120 mg Revacept 5.0±9.3, p=0.918; mean difference 40 mg Revacept vs. Placebo −0.725, 95% CI −6,25-4.8, p=1.00 and 120 mg Revacept vs. Placebo −0.893, 95% CI −6.48-4.7, p=1.00) and after study drug administration (placebo 3.9±9.4, 40 mg Revacept 4.0±11.1, 120 mg Revacept 4.4±7.9, p=0.966; mean difference 40 mg Revacept vs. Placebo 0.046 95% CI −4.78-4,87, p=1.00 and mean difference 120 mg Revacept vs. Placebo 0.471 95% CI −4.38-5.32 p=1.00), for details see Table 2.

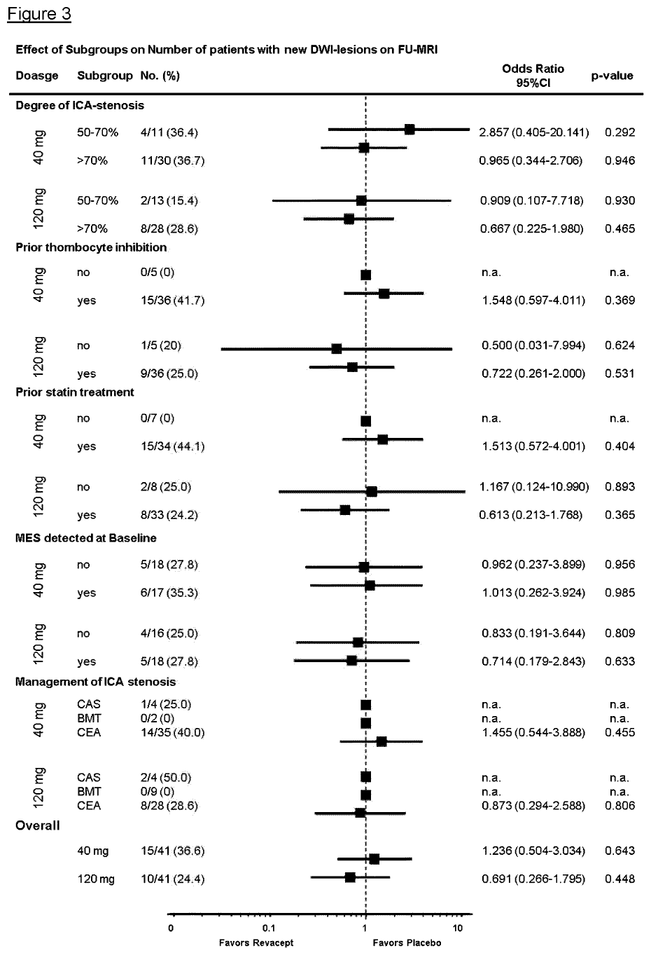

The number of new DWI-lesions decreased from 1.2±2.7 in the placebo group (n=44) to 1.0+2.2 in the 40 mg (n=41) and 0.6+1.7 in the 120 mg group (n=41), reflecting a 50% numerical reduction in the occurrence of new DWI-lesions in the treatment group after application of the higher dosage Revacept; however, significance was not reached (p=0.529, FIG. 2a). This is further demonstrated by a mean difference between 40 mg Revacept and Placebo of −0.201 (95% CI −1.380-0.978, p=1.00) and a mean difference between 120 mg Revacept and Placebo of −0.616 (95% CI −0.1795-0.563, p=0.621). The frequency of patients with new DWI-lesions showed similar rates after treatment with 40 mg Revacept (15/41, 36.6%) compared to Placebo (14/44, 31.8%, Odds Ratio (OR) 1.236, 95% CI 0.504-3.034, p=0.643) and decreased after treatment with 120 mg Revacept (10/41, 24.4%) compared to Placebo (OR 0.691, 95% CI 0.266-1.795, p=0.448, for details see Table 2 and FIG. 3).

The effect in prespecified subgroups (degree of ICA-stenosis, prior thrombocyte inhibition and prior statin treatment) as well as post-hoc analysis (subgroup of patients with MES detected at baseline, management of ICA-stenosis) on frequency of patients with new DWI lesions on follow-up MRI revealed no statistically significant benefit of a specific subgroup (for details see FIG. 3). Nonetheless, in the subgroup of patients with >70% stenosis a more pronounced reduction in number of new DWI-lesion could be observed from 1.1±1.9 in the placebo group to 0.8±1.7 after 40 mg Revacept and 0.5±1.1 after 120 mg Revacept (p=0.407, FIG. 2b). The mean difference in number of new DWI-lesion on follow-up MRI was reduced by −0.421 (95% CI −1.428-0.586, p=0.931) between 40 mg Revacept and Placebo and −0.652 (95% CI −1.677-0.374, p=0.373) between 120 mg Revacept and Placebo.

In a posthoc analysis including only patients with detection of DWI lesions in the vascular territory of the ICA-stenosis number of new DWI-lesions decreased from 1.3 (±3.0) in the Placebo group, to 1.0 (±1.9) after treatment with 40 mg Revacept and to 0.8 (±1.9) after treatment with 120 mg Revacept (p=0.691). For details on prevalence of new DWI-lesions within treatment groups at Baseline and FU please see Table 2.

Concerning functional clinical outcome three months after study drug administration, the NIH-SS showed no evident difference between the three treatment groups (placebo 0 (range 0-1), Revacept 40 mg 0 (0-1), Revacept 120 mg 0 (0-1); Kruskal-Wallis test, p=0.964). Similarly, with regard to NIH-SS at d90 in patients with >70% stenosis, no difference could be observed (placebo NIH-SS 0 (range 0-1), 40 mg Revacept NIH-SS 0 (0-0.3), 120 mg Revacept NIH-SS 0 (0-1); Kruskal-Wallis test, p=0.516).

With regard to clinical efficacy and safety endpoints, statistically not significant differences in distribution between the three treatment groups were observed (for details see Table 3). Occurrence of any stroke at day 90 was 4 patients out of 51 in the placebo group (7.8%) compared to 5 out of 54 patients in the 40 mg Revacept group (9.3%, OR 1.595, 95% CI 0.354-6.920, p=0.555 compared to Placebo) and 2 patients out of 53 (3.8%) in the 120 mg Revacept group (OR 0.601, 95% CI 0.096-3.760, p compared to placebo, p=0.587). The distribution of adverse events and serious adverse events was equally distributed between the three treatment groups (For details see Table 4). In Detail, bleeding complications decreased from 8 out of 51 patients in the placebo group (15.7%) to 6 out of 54 patients in the 40 mg Revacept group (11.1%, OR 0.672, 95% CI 0.216-2.092, p compared to Placebo 0.493) and to 5 out of 53 patients in the 120 mg Revacept group (9.4%, OR 0.560, 95% CI 0.170-1.842, p compared to placebo 0.340). Myocardial infarction or coronary intervention was found to be evenly distributed between the three treatment groups (placebo 3/51 patients (5.9%), 40 mg Revacept 2/54 patients (3.7%), 120 mg Revacept 3/53 patients (5.7%), x², p=0.853). An overview of adverse events is given in supplemental Table 2 and the incidence of serious adverse events is displayed in supplemental Table 3.

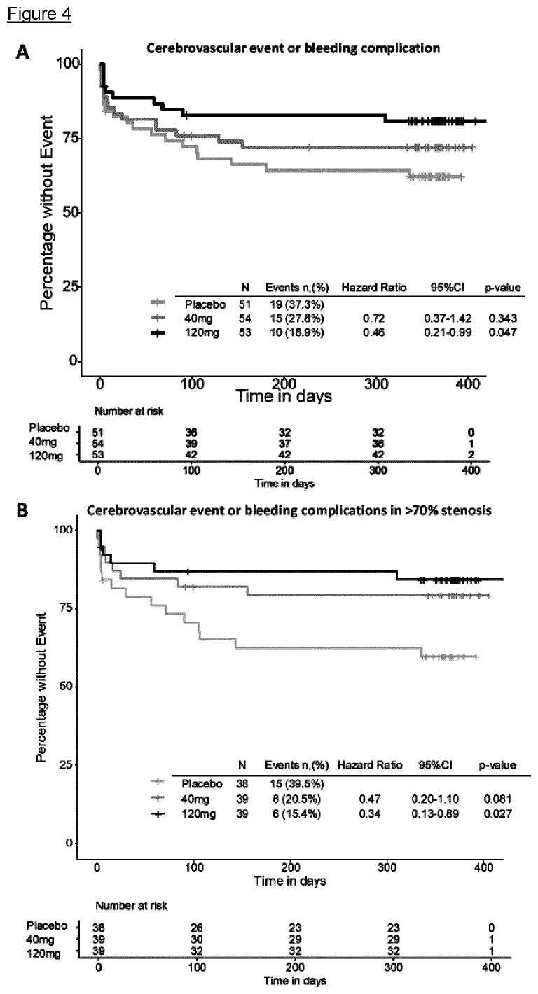

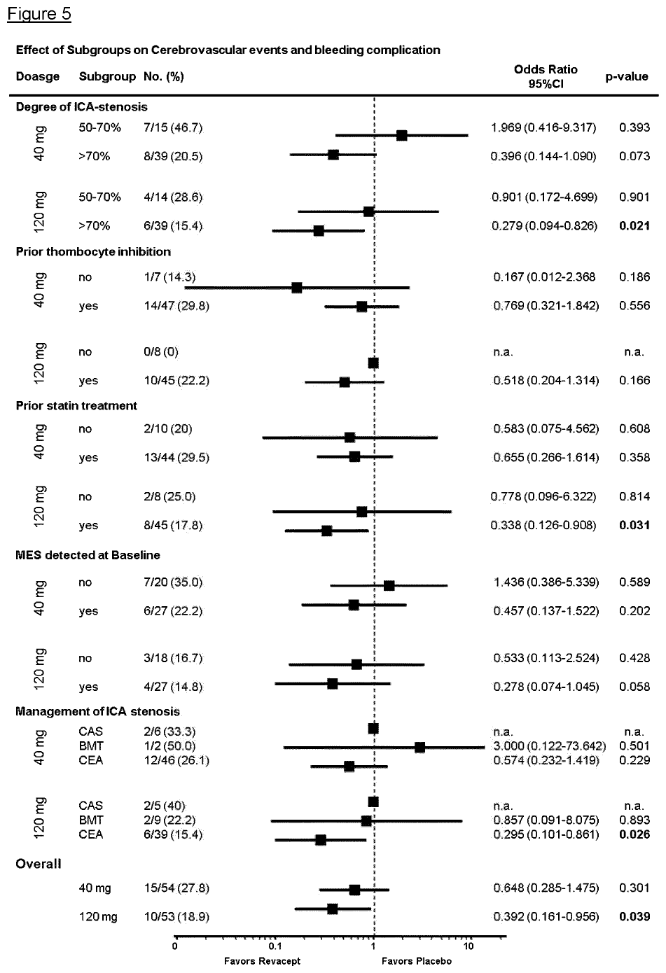

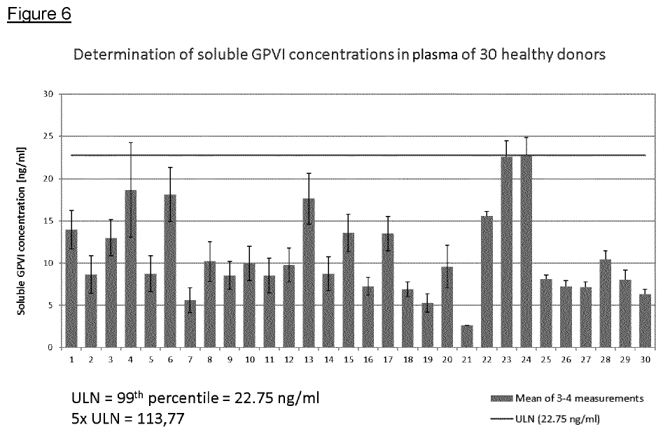

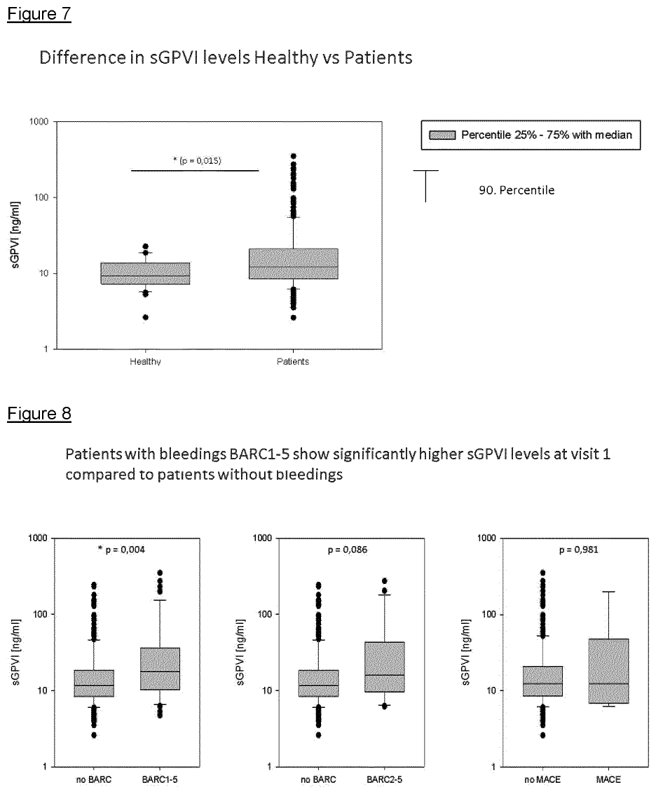

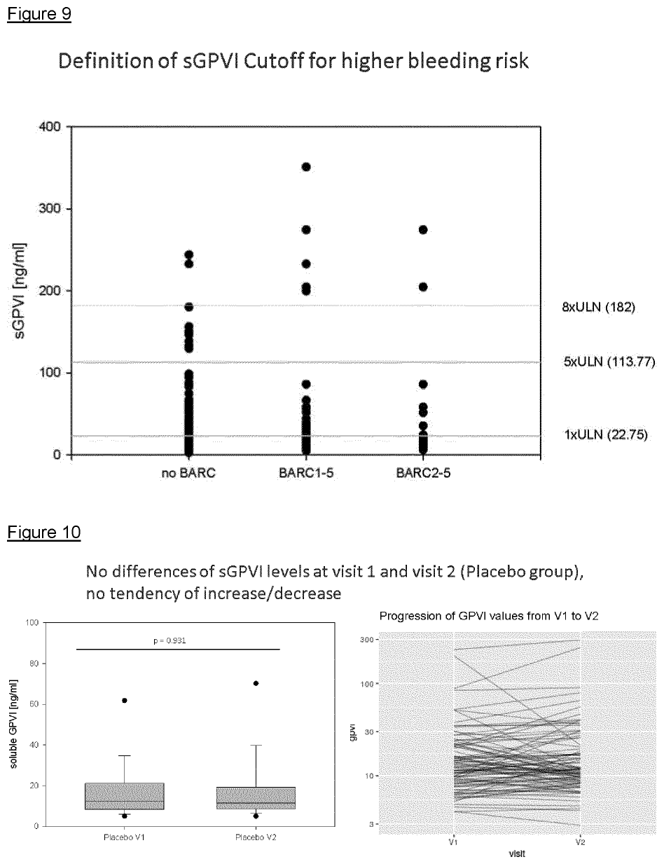

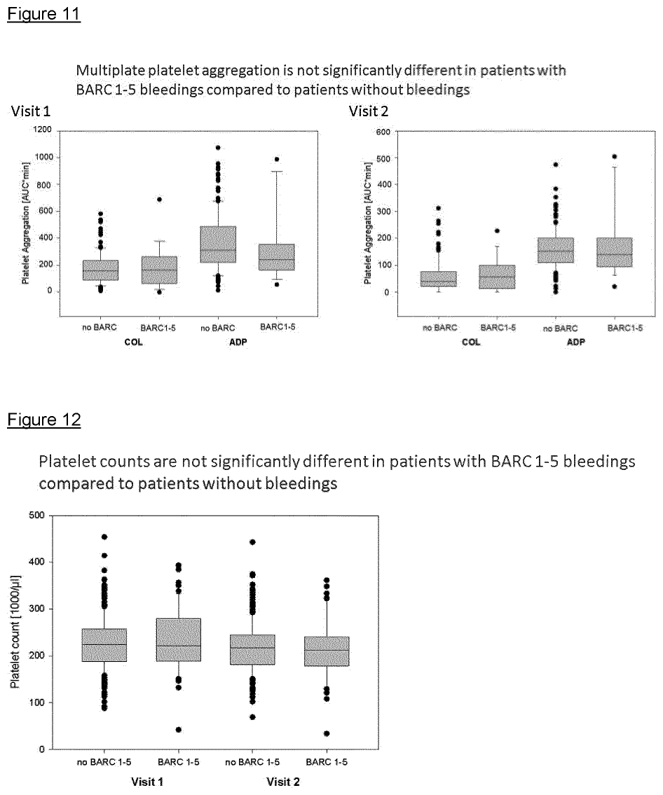

For analysis of efficacy and safety we used the predefined combined endpoint of cerebrovascular events or bleeding complications during the study period. Occurrence of the combined cerebrovascular event or bleeding complication at d90decreased from 16 events (32.4%) in the placebo group, to 13 events (24.1%) after treatment with 40 mg Revacept (OR 0.694, 95% CI 0.294-1.639, p compared to Placebo 0.404) and 9 events (17.0%) after treatment with 120 mg Revacept (OR 0.447, 95% CI 0.177-1.133, p compared to Placebo 0.090). Time-to-event analysis by Cox-regression modeling for time to cerebrovascular event or bleeding complication revealed a 28% risk reduction for treatment with 40 mg Revacept compared to placebo (hazard ratio (HR) 0.72, 95% confidence interval (CI) 0.37-1.42, p=0.343), whereas treatment with 120 mg Revacept resulted in a 54% statistically significant risk reduction compared to placebo (HR 0.46, 95% CI 0.21-0.99, p=0.047, FIG. 4a). Analysis of the subgroup of patients with ICA stenosis >70% (n=116) revealed an even more pronounced difference with Revacept compared to placebo-treatment (for details see FIG. 4b).

The effect of predefined subgroups (degree of ICA-stenosis, prior thrombocyte inhibition and prior statin treatment) as well as post-hoc analysis (subgroup of patients with MES detected at baseline, management of ICA-stenosis) on cerebrovascular events and bleeding complications revealed a potential benefit of patients treated with 120 mg Revacept in the following subgroups: 1) degree of ICA-stenosis above 70% (placebo 15/38 (39.5%) vs. Revacept 120 mg 6/39 (15.4%), OR 0.279 95% CI 0.094-0.826, p=0.021), patients with prior statin therapy (placebo 16/41 (39.0) vs. Revacept 120 mg 8/45 (17.8%), OR 0.338, 95% CI 0.126-0.908, p=0.031), as well as patients undergoing CEA (placebo 16/42 (38.1%) vs. Revacept 120 mg 6/39 (15.4%), OR 0.295, 95% CI 0.101-0.861, p=0.026, for details see FIG. 5).

### Example 6. Diagnostic Method For Selecting Patients For Preventing Bleeding Complications

A clinical trial called “Revacept, a novel inhibitor of platelet adhesion in patients with stable coronary artery disease undergoing elective percutaneous coronary interventions: A phase II, multicenter, randomized, dose-finding, double-blind and placebo-controlled study” (EudraCT Number: 2015-000686-32) was conducted according to the study protocol Revacept/CAD/02.

### Methods:

GPVI is sheded from the surface of platelets upon activation after plaque-mediated platelet activation into the blood. With an ELISA-based assay we determined the amount of soluble GPVI in patients with stable CAD undergoing elective PCI.

Samples for the biomarker study, platelet aggregation and platelet count were taken before and after study drug application, patients underwent coronary intervention and major clinical events were recorded for 30 days.

Results: 332 patients with stable CAD treated with aspirin (100-300 mg daily) and clopidogrel (600 mg loading dose followed with 75 mg daily) were included into a randomized controlled trial, the Revacept Plaster study. Additionally, patients were randomized for placebo, Revacept 80 mg and 160 mg on top of the basal antiplatelet therapy.

### Results: See FIG. 6-12

### Patients Clinical Outcome:

Overall, 334 patients were included into the study and 332 received the study medication. 93 patients received placebo, 119 80 mg Revacept (2 patients were excluded for study medication after screening) and 120 patients 160 mg Revacept. The mean age was 67 years with more than 75% male patients with the typical risk factors for atherosclerosis. 55% had femoral access for cardiac catheterization and 45% radial or brachial. All patients had negative troponin T values to confirm the stable CAD. All patients received standard dual anti-platelet therapy with aspirin and clopidogrel. 92% were on aspirin and 54% clopidogrel at baseline. 72% received additional aspirin loading (300 mg) and 82% additional clopidogrel loading (600 mg) before cardiac intervention.

The clinical events such as ischemic complications (MACE) were recorded in 9 patients and BARC bleeding grade 1-5 in 53 patients, and BARC 2-5 bleeding in 21 patients within the observation period of 30 days after PCI.

### High Sensitivity Troponin T:

Baseline levels for TnT were within the normal range at screening (11 ng/L). In patients with no MACE (n=325) TnT increased to 30.5 (17.3-62.5) ng/L and in patients with MACE (n=9) increased to 463 (37.0-1342.5) ng/L after the intervention (p=0.002). The correlation for peak TnT to MACE was highly significant with p=0.00005. As expected, the correlation to BARC bleeding was neglectable. Soluble GPVI:

30 healthy volunteers (age 28-65 years) were investigated to determine the baseline sGPVI level with 9.1 (7.3-13.7) ng/ml (Median and 25-75% interval). As the upper limit of normality, we chose the 99th percentile from this healthy control population (22.8 ng/ml). In 318 patients with stable CAD sGPVI levels were significantly (p=0.015) increased to 12.2 (8.5-21.1) ng/ml at V1. There was no difference in the sGPVI levels at V2 in the placebo group (n=93, V2 11.6 (8.7-19.3) ng/ml). As the sGPVI assay interferes with the recombinant GPVI/Revacept, V2values in the Revacept groups were massively increased due to the study drug. Therefore, all further correlations were calculated from the V1 of the entire study population. In patients experiencing MACE (n=8) the sGPVI levels at V1 (12.3 (6.9-47.8) ng/ml) were not different to those without MACE (n=310, 12.2 (8.5-20.9) ng/ml). The correlation for MACE was p=0.98. In contrast, in patients with BARC 1-5 bleedings (n=53) sGPVI levels were significantly (p=0.004) increased to 17.9 (10.3-36.4) ng/ml compared to patients without bleeding (sGPVI 11.6 (8.3-18.6) ng/ml). In patients with BARC 2-5 bleedings (n=21) the difference to patients without bleeding were not significant (sGPVI 15.9 (9.6-43.5) ng/ml, p=0.09). As cutoff values (upper limit of normal, ULN) for the prognosis of future bleedings the 99^{th} percentile of the healthy population with 22.8 mg/mL was chosen and the 5×ULN (113.8 mg/mL) and 8×ULN (182.0 mg/mL). The odds ratio (OR) to experience future bleeding complications with sGPVI at ULN was 2.4 and for 5×ULN 2.7 and increased dramatically to 13.7 with 8×ULN. sGPVI values correlated for logistic regression with p=0.004 for BARC 1-5 bleedings. For BARC 2-5 bleedings the logistic regression correlation was 0.02 and the OR was 2.8 for the 5×ULN and 14.6 for the 8×ULN. Sensitivity and specificity, the positive predictive values and negative predictive value for BARC 1-5 and BARC 2-5 bleedings is shown in table 1.

### Platelet Aggregation:

Overall, 168 patients were studied with the multiplate platelet aggregation method. Platelet aggregation was already substantially reduced both for ADP-mediated aggregation and collagen-mediated aggregation at V1 (after clopidogrel and aspirin loading) and even more at V2 48 h after application of study drug. Collagen-mediated platelet aggregation was numerically higher at V2 in patients with BARC 1-5 bleeding (56.0 (12.8-99.8) AUC*min) compared to no bleeding (38.5 (21.0-75.8) AUC.*min) but far from significant (p=0.73). The ADP-mediated aggregation was almost similar in BARC 1-5 bleeding (140.0 (95.0-200.3) AUC*min) compared to no bleeding (152.0 (109.8-200.5) AUC*min).

The correlation for future bleeding events for BARC 1-5 bleeding at the V1 visit (after ASS/clopidogrel, before study drug) was only poor with p values for collagen-mediated aggregation of 0.92 and for ADP-mediated aggregation of 0.25 (logistic regression). For more severe bleeding of BARC 2-5 the logistic regression was 0.72 and 0.15. At V2 48 h after loading and study drug the correlation was still poor for BARC 1-5 (p values 0.71 and 0.59) and 0.15 for collagen-mediated and at the border of significance for ADP mediated aggregation (p=0.05) for BARC 2-5 bleedings.

### Platelet Count:

There was also no difference between platelet counts in patients with BARC 1-5 bleeding either at V1 (221,000 (189,000-278,750)/μl) compared to no bleeding (224,500 (188,000-256,750)/μl) nor at V2 (BARC 1-5 211,500 (178,250-240,500)/μl and no bleeding 216,500 (181,000-245,000)/μl).

There was no correlation between the sGPVI levels neither at V1 or V2 to the collagen nor to the ADP-mediated platelet aggregation. Moreover, there was no correlation of sGPVI levels to platelet counts either at V1 nor at V2.

### CONCLUSION

sGPVI determination could help to personalize the anti-platelet therapy of patients undergoing PCI before the intervention takes place.

In patients with acute coronary syndrome GPVI receptor density on platelets was shown to be increased as an early marker for ischemia.

1. A pharmaceutical composition for parenteral application, comprising
(a) more than 2.4 mg/ml of a collagen-binding dimeric fusion protein comprising an extracellular domain of glycoprotein VI, the fusion protein having an amino acid sequence of SEQ ID NO: 1, or an amino acid sequence that is at least 99% homologous to the amino acid sequence of SEQ ID NO: 1; and
(b) a pharmaceutically acceptable buffer, the buffer comprising
(b1) a buffering component in combination with a pH adjusting agent, which is 10 mM His/HCl, and
(b2) mannitol as a stabilizing agent in an amount of 4 percent by weight based on the total weight of the pharmaceutical composition and
(b3) saccharose in an amount of 2.5 percent by weight based on the total weight of the pharmaceutical composition as a stabilizing agent, and
which has a pH of 7.

2. The pharmaceutical composition for parenteral application according to claim 1, wherein the buffer further comprises
(b4) a detergent, preferably in an amount of 0.05 percent by weight based on the total weight of the pharmaceutical composition.

3. The pharmaceutical composition for parenteral application according to claim 2, wherein the detergent is selected from Tween 20, Tween 40, or Tween 80.

4. The pharmaceutical composition for parenteral application according to claim 1 which is storage stable at a temperature of 8° C. for at least 6 months; and/or
which is an aqueous dispersion; and/or
which is packaged in a unit dose perfusion syringe containing at most 50 ml of the composition.

5. A lyophilized composition adapted to provide a pharmaceutical composition for parenteral application as defined in claim 1 after hydration, which lyophilized composition is preferably stoarge stable at 25° C. for at least 6 months.

6. A pharmaceutical composition according to claim 1 for use in the prevention or treatment of bleeding complications in a patient by administering the pharmaceutical composition to a patient selected based on a soluble glycoprotein VI concentration in blood plasma of more than 22.8 ng/ml.

7. The pharmaceutical composition for use according to claim 6, wherein the patient is a patient suffering from thrombotic complications;
preferably wherein the patient suffering from thrombotic complications is a patient suffering from stroke, myocardial infarction or peripheral artery disease; and/or
wherein the patient is a trauma patient, a transplant patient, a cancer patient, a patient with idiopathic thrombocytopenia purpura (ITP) or immune thrombocytopenia and/or a patient suffering from cardiovascular disease; and/or wherein the bleeding complications are postoperative bleeding complications;
and/or wherein the dimeric fusion protein is administered prior to a percutaneous intervention or surgery; and/or
wherein the patient receives additional standard anti-platelet therapy or anti-coagulant therapy; and/or
wherein
wherein the dimeric fusion protein is administered intravenously at a dose of from 5 to 300 mg; and/or
which is administered as a single dose or multiple doses.

8. Kit-of-parts comprising a pharmaceutical composition defined in claim 1, and a diagnostic antibody directed against soluble glycoprotein VI.

9. A diagnostic method for selecting a patient benefitting from administration of a dimeric fusion protein comprising an extracellular domain of glycoprotein VI for preventing bleeding complications, which method comprises
(a) determining a soluble GPVI concentration in blood plasma of the patient; and
(b) selecting the patient if the soluble GPVI concentration is above 22.8 ng/ml.

10. The diagnostic method according to claim 9, wherein the patient is to be treated by percutaneous intervention or surgery; and/or preferably which is computer-implemented.
